(19) United States
(12) Patent Application Publication
(10) Pub. No.: US 2024/0361755 A1
(43) Pub. Date: Oct. 31, 2024

(54) SWIMMING POOL MONITORING SYSTEMS AND METHODS

(71) Applicant: PoolPal Corp., Markham (CA)

(72) Inventor: Mehdi Pahlavan, Markham (CA)

(21) Appl. No.: 18/649,312

(22) Filed: Apr. 29, 2024

Related U.S. Application Data
(60) Provisional application No. 63/462,310, filed on Apr. 27, 2023.

(51) Int. Cl. G05B 19/416; G01D 21/02; G01N 33/18; G01N 35/10

(52) U.S. Cl. CPC G05B 19/416; G01D 21/02; G01N 33/1886; G01N 35/10; G05B 2219/37371

(57) ABSTRACT

A system for pool monitoring comprises a plurality of sensors, each sensor being configured to monitor a parameter of a pool; a control unit operatively connected to the plurality of sensors, the control unit being configured to determine a status of water in the pool based on a signal received one or more of the sensors, and a cloud computer operatively connected to the control unit, the cloud computer configured to adjust a plurality of settings of the control unit; and an end node computer operatively connected to the cloud computer, the cloud computer being configured to transmit information received from each sensor and the status determined by the control unit to the end node computer for display.

### TECHNICAL FIELD

The disclosed exemplary embodiments relate to methods and systems for monitoring of a swimming pool.

### BACKGROUND

Commercial swimming pools are a widely available amenity, used by many in various settings including for example, in condominiums, sport and/or community centers, and hotels. The water quality and maintenance of swimming pools is important for both the wellbeing of those using the pool, and for the pool itself, while being heavily regulated in the USA and Canada. In some cases, water quality and pool maintenance may be manually conducted, with pool operators and custodians responsible for following a set schedule for monitoring various parameters associated with the pool and the water contained within it.

### SUMMARY

The following summary is intended to introduce the reader to various aspects of the detailed description, but not to define or delimit any invention.

Described herein are systems, and associated methods, for pool monitoring, control and prediction. In one embodiment, the system may comprise: a control unit; a plurality of sensors, operatively coupled to the control unit, each sensor of the plurality of sensors configured to monitor a parameter of a pool; an end node computer, operatively coupled to the control unit, the end node computer configured to adjust a plurality of settings of the control unit; and a cloud computer operatively connected to the end node computer; wherein the control unit is configured to determine a status of water, water level, temperature, clarity, chemistry, pool occupancy in various zones, ambient air temperature, relative humidity, pump and filter pressures, amperage draw, water speed (flow) in mechanical pipes, potable make-up water metering, pool water waste metering, in the pool based on a signal from each of the plurality of sensors; and wherein the end node computer transmits information received from each sensor and the status determined by the control unit to the cloud computer.

Accordingly, in at least one broad aspect, there is provided a system for pool monitoring, the system comprising: a plurality of sensors, each sensor being configured to monitor a parameter of a pool; a control unit operatively connected to the plurality of sensors, the control unit being configured to determine a status of water in the pool based on a signal received one or more of the sensors, and a cloud computer operatively connected to the control unit, the cloud computer configured to adjust a plurality of settings of the control unit; and an end node computer operatively connected to the cloud computer, the cloud computer being configured to transmit information received from each sensor and the status determined by the control unit to the end node computer for display.

In some cases, the plurality of sensors comprises at least two of: a water level sensor, a bin level sensor, a load cell sensor, a pump rpm sensor, a vibration sensor, a pressure sensor, a leak sensor, a water meter, a current meter, an ambient temperature sensor, and a relative humidity sensor.

In some cases, the system further comprises a chemical monitoring unit, wherein the control unit is configured to determine the status of water in the pool based on information from the chemical monitoring unit, and wherein the cloud computer transmits information received from the chemical monitoring unit to the end node computer for display.

In some cases, the chemical monitoring unit comprises a pH sensor, an Oxidization Reduction Potential (ORP) sensor, a temperature sensor, a turbidity sensor, a total dissolved solids (TDS) sensor, and a water flow sensor.

In some cases, the control unit is operatively connected to a water pump, the pump rpm sensor and the water meter, the control unit being configured to control the water pump based on a signal received from the pump rpm sensor and the water meter.

In some cases, the control unit is operatively connected to the water level sensor and the leak sensor, the control unit being configured to determine a rate of water loss based on a signal from the water level sensor and the leak sensor.

In some cases, the system further comprises an autonomous testing unit configured to test the water based on a predetermined schedule, and wherein the control unit is configured to determine the status of water in the pool based on information from the autonomous testing unit, and wherein the end node computer is configured to transmit information received from the autonomous testing unit to the cloud computer

In some cases, the system further comprises a machine learning component that, based on the transmitted information, is configured to generate a maintenance schedule.

In another broad aspect, there is provided a method of pool monitoring, the method comprising: providing a plurality of sensors, each sensor of the plurality of sensors being configured to monitor a parameter of a pool; determining, by a control unit operatively connected to the plurality of sensors, a status of water in the pool based on a signal from each of the plurality of sensors; transmitting, by the control unit, information received by the control unit from the plurality of sensors to a cloud computer; and receiving, from the cloud computer, a maintenance schedule based on the transmitted information.

In some cases, the method further comprises monitoring, by a chemical monitoring unit, a level of a chemical in the pool; determining, by the control unit, the status of water in the pool based on information from the chemical monitoring unit; and transmitting, by the control unit, the information from the chemical monitoring unit to the cloud computer.

In some cases, the method further comprises controlling, by the control unit, a water pump based on a signal from a pump rpm sensor and a water meter.

In some cases, the method further comprises, determining, by the control unit, a rate of water loss based on a signal from the water level sensor and the leak sensor.

In some cases, the method further comprises carrying out testing, by an autonomous testing unit, the water in the pool based on a predetermined schedule; determining, by the control unit, the status of water in the pool based on information from the autonomous testing unit; and transmitting, by the control unit, the information from the autonomous testing unit to the cloud computer.

In some cases, wherein the maintenance schedule is generated by a machine learning component.

In another broad aspect, there is provided a system for pool chemical monitoring comprising: a housing; a plurality of sensors, each sensor of the plurality of sensors being configured to monitor a parameter of a pool; a water flow monitor; a processor, configured to receive a signal from each of the plurality of sensors and the water flow monitor to determine a status of water in the pool; and a display, configured to receive the status from the processer and provide an indication of the status.

In some cases, the plurality of sensors comprises at least a pH sensor, an Oxidization Reduction Potential (ORP) sensor, a temperature sensor, a total dissolved solids (TDS) sensor, a turbidity sensor, and a water flow sensor.

In some cases, the housing is configured to receive a flow of water from the pool, the plurality of sensors being in fluid communication with the flow of water.

In some cases, the system further comprises a pH pump controller, operatively coupled to a pH pump.

In some cases, the pH pump controller is configured to control the pH pump based on a pH setpoint, a pH run mode, and a signal from the pH sensor.

In some cases, the system further comprises an ORP controller, operatively coupled to an ORP pump.

In some cases, the ORP controller is configured to control the ORP pump based on an ORP setpoint, an ORP run mode, and a signal from the ORP sensor.

In some cases, the system further comprises at least one alarm corresponding to the plurality of sensors, wherein the at least one alarm comprises at least one of an audio and a visual indicator.

In some cases, the processor is configured to transmit information on the signal from each sensor and the at least one alarm to a device.

In some cases, the water flow monitor comprises a flow switch, wherein the flow switch is configured to adjust the flow of water based on a status of the at least one alarm.

In another broad aspect, there is provided a method for pool chemical monitoring comprising: providing a plurality of sensors, each sensor of the plurality of sensors configured to monitor a parameter of a pool; determining, by a processor, a status of water in the pool based on a signal from each of the plurality of sensors; and displaying an indication of the status.

In some cases, the method further comprises controlling a pH pump based on a pH setpoint, a pH run mode, and a signal from the pH sensor.

In some cases, the method further comprises controlling an ORP pump based on an ORP setpoint, an ORP run mode, and a signal from the ORP sensor.

In some cases, the method further comprises generating at least one alarm corresponding to the plurality of sensors, wherein the at least one alarm comprises at least one of an audio and a visual indicator.

In another broad aspect, there is provided an apparatus for autonomous water testing in a pool comprising: a sample vessel situated in a moveable housing; a plurality of valves, each valve coupled to a reagent supply or a water supply from the pool, and a flow meter; and a microcontroller configured to control movement of the housing, a flow of reagent or water through the plurality of valves, and perform the water testing based on a predetermined schedule.

In some cases, the plurality of valves comprises solenoid valves.

In some cases, the microcontroller is configured to move the housing so that the sample vessel is in fluid communication with a source of tap water for washing the sample vessel.

In some cases, the microcontroller is configured to move the housing so that the sample vessel is in fluid communication with a reagent supply and a sample of water from the water supply to mix the sample with a reagent from the reagent supply.

In some cases, the microcontroller is configured to control an amount of liquid deposited in the sample vessel based on the flow meter.

In some cases, the reagent supply is coupled to an air compressor.

In some cases, the apparatus further comprises a camera configured to capture an image of the sample vessel when it contains the reagent and the sample.

In some cases, the microcontroller is configured to process the image to test for at least one of free available chlorine (FAC), total available chlorine (TAC), combined chlorine (CC), acidity and alkalinity of the sample.

In another broad aspect, there is provided a method for autonomous water testing in a pool comprising: washing a sample vessel on a housing to with a source of tap water; moving the sample vessel to fluidly communicate with a source of reagent; depositing an amount of a reagent into the sample vessel; moving the sample vessel to fluidly communicate with a source of pool water; depositing an amount of pool water into the sample vessel; processing a result of mixing the amount of pool water with the amount of reagent with a microcontroller; and determining a status of the pool water based on the result.

In some cases, the depositing the amount of the reagent comprises operating, by the microcontroller, a valve.

In some cases, the depositing the amount of pool water comprises operating, by the microcontroller, another valve.

In some cases, the microcontroller performs the autonomous testing based on a predetermined schedule.

In some cases, the processing the result comprises capturing an image of the mixed pool water and reagent by the microcontroller.

In some cases, the processing the result further comprises analysing the captured image to determine at least one of free available chlorine (FAC), total available chlorine (TAC), combined chlorine (CC), acidity and alkalinity of the pool water.

In another broad aspect, there is provided a system for pool monitoring comprising: at least one camera having a field of view of a pool and an area surrounding the pool; an indicator disposed on a bottom surface of the pool; a skimmer disposed on a wall of the pool; and a processor operatively connected to the at least one camera and the alarm, the processor being configured to receive images of the pool captured by the at least one camera and determine one or more of: at least one water parameter based on the received images with respect to at least one of the indicator and the skimmer; an occupancy of the pool; an occupancy of the area surrounding the pool; and indications of pre-drowning of at least one occupant of the pool.

In some cases, the area surrounding the pool is divided into a plurality of deck occupancy zones.

In some cases, wherein the processor is further configured to determine an occupancy of each of the plurality of deck occupancy zones.

In some cases, the pool is divided into one or more water occupancy zone.

In some cases, the processor is further configured to determine an occupancy of the one or more water occupancy zone.

In some cases, the system further comprises a cloud computer, and wherein the processor is further configured to send data relating to the at least one water parameter, the occupancy of the pool, and the occupancy of the area surrounding the pool to the cloud computer for further processing.

In some cases, the data comprises periodic data and event data.

In some cases, the alarm is at least one of an audible alarm and a visual alarm.

In some cases, the at least one water parameter includes a water level and a water clarity.

In some cases, the processor determines the at least one water parameter based on a level of the water with respect to the water skimmer.

In some cases, the processor determines the at least one water parameter based on a visibility of the indicator.

In some cases, the at least one camera includes a thermal camera configured to detect a temperature of the pool.

In another broad aspect, there is provided a method of pool monitoring, comprising: obtaining images of a pool and an area surrounding the pool from at least one camera; determining at least one water parameter based on the images; determining an occupancy of the pool and an occupancy of the area surrounding the pool based on the images; identifying pre-drowning indicators, if any; and if any pre-drowning indicators are identified, activating an alarm.

In some cases, the method further comprises dividing the area surrounding the pool into a plurality of deck occupancy zones.

In some cases, the method further comprises determining an occupancy of each of the plurality of deck occupancy zones.

In some cases, the method further comprises dividing the pool into one or more water occupancy zone.

In some cases, the method further comprises determining an occupancy of the one or more occupancy zone.

In some cases, the method further comprises sending data relating to the at least one water parameter, the occupancy of the pool, and the occupancy of the area surrounding the pool to a cloud computer for further processing.

In some cases, the data comprises periodic data and event data.

In some cases, the activating the alarm comprises activating at least one of an audible alarm and a visual alarm.

In some cases, the at least one water parameter includes a water level and a water clarity.

In some cases, the determining at least one water parameter comprises determining the at least one water parameter based on a level of the water with respect to a water skimmer on a wall of the pool.

In some cases, the determining at least one water parameter comprises determining the at least one water parameter based on a visibility of an indicator on a bottom of the pool.

In some cases, the method further comprises determining a temperature of the pool based on data received from a thermal camera.

According to some aspects, the present disclosure provides a non-transitory computer-readable medium storing computer-executable instructions. The computer-executable instructions, when executed, configure a processor to perform any of the methods described herein.

### BRIEF DESCRIPTION OF THE DRAWINGS

The drawings included herewith are for illustrating various examples of articles, methods, and systems of the present specification and are not intended to limit the scope of what is taught in any way. In the drawings:

FIG. 1 is a schematic diagram of an exemplary system for swimming pool monitoring in accordance with at least some embodiments;

FIG. 2 is a schematic block diagram of the exemplary system of FIG. 1;

FIG. 3 is a block diagram of a computer in accordance with at least some embodiments;

FIG. 4 is a flowchart diagram of an exemplary method for swimming pool monitoring for execution by the system of FIG. 2;

FIG. 5 is a flowchart diagram of additional steps of the exemplary method of FIG. 4; and

FIG. 6 is a schematic block diagram of an exemplary system for swimming pool chemical monitoring in accordance with at least some embodiments;

FIG. 7 is a flowchart diagram of an exemplary method for swimming pool chemical monitoring for execution by the system of FIG. 6;

FIG. 8 is a schematic block diagram of an exemplary apparatus for autonomous water testing in accordance with at least some embodiments;

FIG. 9 is a flowchart diagram of an exemplary method for autonomous water testing for execution by the system of FIG. 8; and

FIG. 10A is a schematic view of a system for monitoring in accordance with at least one embodiment;

FIG. 10B is a perspective view of a portion of the system of FIG. 10A;

FIG. 11 is a block diagram of the system of FIG. 10A;

FIG. 12A to FIG. 12E are examples of pool zone representations for a variety of different pool shapes used with the system of FIG. 10A;

FIG. 13 is a view of a hot tub or spa used with the system of FIG. 10A;

FIG. 14 is a flowchart diagram of an exemplary method for pool monitoring for execution by the system of FIG. 10A;

FIG. 15A is an example of a graphical user interface dashboard displayed by the system of FIG. 1;

FIGS. 15B and 15C are exemplary dashboard gauges and dials for use with the graphical user interface dashboard of FIG. 15A;

FIG. 16 is an example of a graphical user interface dashboard associated with the autonomous testing unit of FIG. 8, and displayed by the system of FIG. 1;

FIGS. 17A and 17B are examples of display interfaces for a hot tub and a pool respectively, for use with the graphical user interface dashboard of FIG. 15A;

FIGS. 18A and 18B are examples of adjustable settings pages presented by the graphical user interface dashboard of FIG. 15A;

FIGS. 19A, 19B, and 19C are examples of charts pages presented by the graphical user interface dashboard of FIG. 15A;

FIG. 20 is an example notification log page presented by the graphical user interface dashboard of FIG. 15A;

FIG. 21 is an example data log page presented by the graphical user interface dashboard of FIG. 15A;

FIG. 22 is an example log sheet page presented by the graphical user interface dashboard of FIG. 15A;

FIG. 23 is an example daily checklist presented by the graphical user interface dashboard of FIG. 15A;

FIGS. 24A and 24B are examples of safety inspection record pages presented by the graphical user interface dashboard of FIG. 15A;

FIG. 25 is an example of a monitoring log page presented by the graphical user interface dashboard of FIG. 15A;

FIG. 26 is an example of an alarm log page presented by the graphical user interface dashboard of FIG. 15A; and

FIG. 27 is an example of an version history viewable page presented by the graphical user interface dashboard of FIG. 15A.

### DETAILED DESCRIPTION

The upkeep of swimming pools requires water monitoring to ensure that the water is safe for use, and has safe levels of chemicals for sanitization of the water. The chemicals used may be chlorine, bromine, or ozone for example. Excess levels of such chemicals may result in skin or eye irritation for users, while too low a level of these chemicals may have ineffective sanitizing results. Therefore it is important that these chemicals remain at an appropriate level in the water.

Regular pool testing includes testing the Free Available Chlorine (FAC), Total Available Chlorine (TAC), Combined Chlorine (CC), acidity and alkalinity of the water. These tests are important for ensuring the correct pathogen deactivation action of disinfectant present in the pool water. An unbalanced pH can result in staining or scale formation in the pool, corrosion of metal accessories, and can impact the effectiveness of the disinfectant. The FAC, TAC, and CC should be tested regularly to ensure there is a balanced water chemistry. The FAC, TAC, and CC can be affected by sunlight, temperature, acidic rain showers, and wind.

Conventional water quality monitoring approaches have relied on manually testing the water at set intervals and adjusting the chemicals applied to the water in the pool. This relies on a pool operator or custodian being available and following the required schedule, with water testing results gathered and stored manually.

Similarly, the maintenance of pool components may follow a set schedule with the operator or custodian manually checking components, with little indication of when a component may fail. Components may be pool pumps, filters, tubing, etc. Maintenance is therefore reactive and may result in downtime of the pool.

Automation, when applied to pool monitoring, may result in more efficient water quality and pool component monitoring, with maintenance becoming proactive based on the outputs of automated monitoring.

The described embodiments provide systems and methods for pool monitoring, for both water quality and pool components.

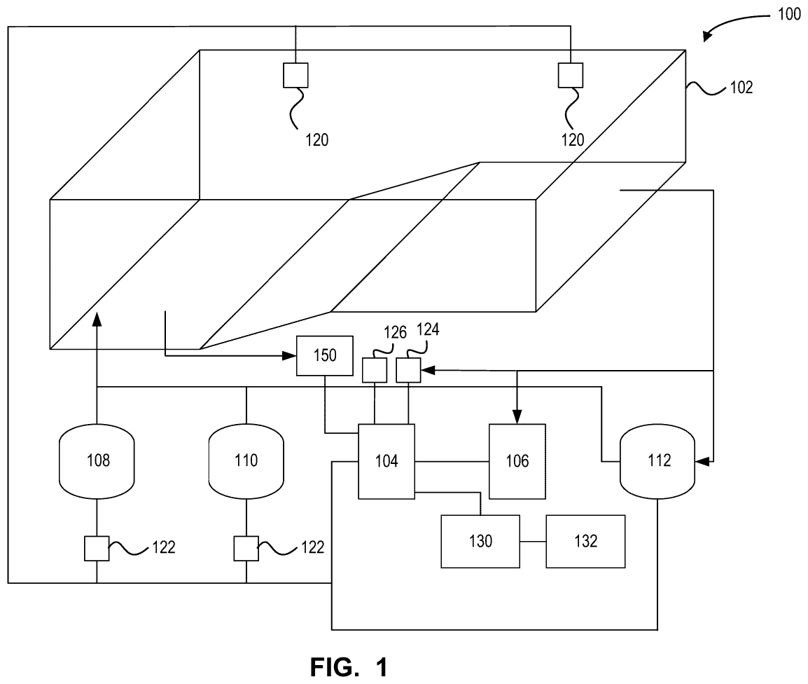

Referring now to FIG. 1, there is illustrated an exemplary system 100 for swimming pool monitoring in accordance with at least some embodiments The system 100 is generally configured to monitor and operate a swimming pool 102, and operable components associated with the swimming pool 102. In the example shown, the system 100 comprises a control unit 104, a chemical monitoring unit 106, a chemical bin 108, an acid bin 110, a pump 112, water level sensors 120, bin level sensors 122, a water meter 124, an ambient temperature and relative humidity sensor 126, a cloud computer 130, an end-node computer 132, and an autonomous testing unit 150.

The pump 112 is in fluid communication with the pool 102, the chemical bin 108, and the acid bin 110. The pump 112 may be a conventional pool pump, as known in the industry, and is configured to circulate water through the pool's water treating systems, described below, as well as through the pool's heater (not shown) and filter not shown). The pump 112 is operatively connected to the control unit 104. The chemical bin 108 and the acid bin 110 are each connected to a respective bin level sensor 122. The bin level sensors 122 are operatively connected the control unit 104. The chemical monitoring unit 106 and the autonomous testing unit 150 are in fluid communication with the pool 102. The water meter 124 is in fluid communication with the pool 102. The water level sensors 120, the water meter 124, and the ambient temperature and relative humidity sensor 126 are operatively connected to the control unit 104. The control unit 104 and the end node computer 132 are operatively connected to the cloud computer 130.

Although the system 100 is described in the context of a pool 102, other configurations are possible. In some cases, the system 100 may alternatively be used in conjunction with a hot tub or a spa.

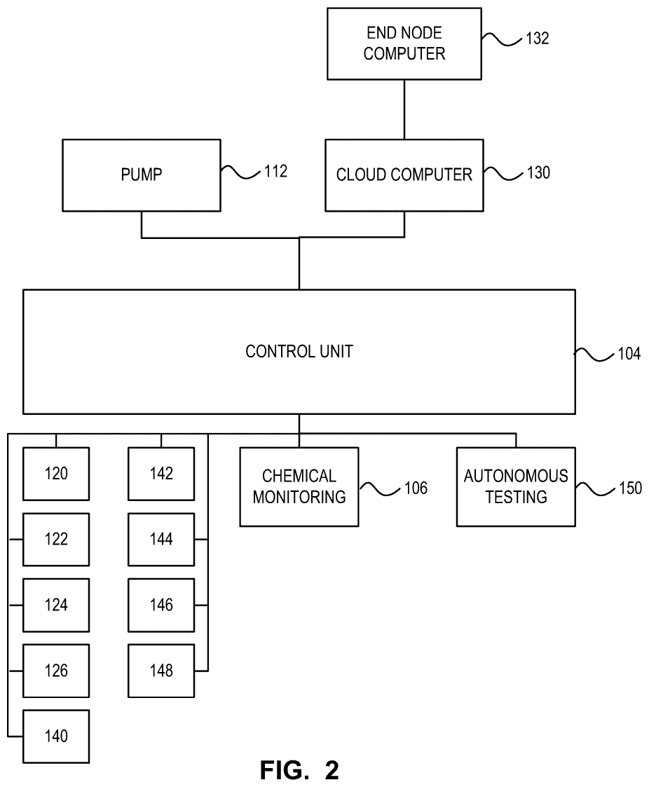

Referring now to FIG. 2, there is illustrated a schematic block diagram of a monitoring portion of the system 100. As can be seen, the control unit 104 is centrally and operatively coupled to the chemical monitoring unit 106, the pump 112, the water level sensor 120, the bin level sensor 122, the water meter 124, the ambient temperature and relative humidity sensor 126, the cloud computer 130, the end node computer 132, a pump revolutions per minute (rpm) sensor 140, a current meter 142, a vibration sensor 144, a pressure sensor 146, a leak sensor 148, and the autonomous testing unit 150. The control unit 104 may be a microcontroller, a programmable logic controller (PLC), a processor, or the like.

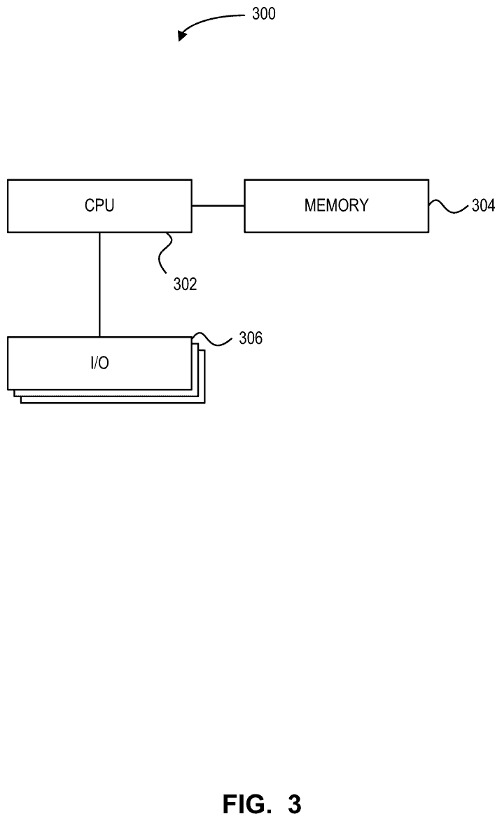

Referring now to FIG. 3, there is illustrated a simplified block diagram of a computer in accordance with at least some embodiments. Computer 300 is a generic example of a computer, such as the end node computer 132 or the cloud computer 130 of FIG. 2. Computer 300 generally has at least one processor 302 operatively coupled to at least one memory 304, and at least one additional input/output device 306.

The at least one memory 304 includes a volatile memory that stores instructions executed or executable by processor 302, and input and output data used or generated during execution of the instructions. Memory 304 may also include non-volatile memory used to store input and/or output data along with program code containing executable instructions.

Processor 302 may transmit or receive data via a data communications interface (not shown), or may also transmit or receive data via any additional input/output device 306 as appropriate.

Turning again to FIG. 1, the water flow layout used by the system 100 may be better seen. The pump 112 is configured to provide a suction force to the pool 102 and to extract water therefrom for treatment. The water extracted by the pump 112 is then treated by a chemical and an acid, which are stored in the chemical bin 108 and the acid bin 110 respectively, and then returned to the pool 102. Although only one chemical bin 108 and only one acid bin 110 are shown, there may alternatively be more than one of each bin included in the system 100. Examples of chemicals and acids used include, but are not limited to, sodium bicarbonate, muriatic acid, bromine, sodium hypochlorite, calcium hypochlorite slurry solution, chlorine and bromine pucks. The pH in the pool 102 may be controlled by a base and an acid, such as sodium bicarbonate and muriatic acid. Sanitizers used in the pool 102 may be bromine, sodium hypochlorite, calcium hypochlorite slurry solution. Chlorine and bromine pucks may be used in an erosion feeder (not shown).

The bin level sensors 122, which are respectively coupled to the chemical bin 108 and the acid bin 110, are each configured to detect the content of the chemical bin 108 or the acid bin 110, to determine an amount of chemical or acid remaining therein. In the example shown, the bin level sensors 108 are positioned generally below each of the chemical bin 108 and the acid bin 110, such as at the base of the bin, and are configured to sense a weight, level, or volume of the content of the bin. In particular, the bin level sensors 122 are configured to each wirelessly send the information relating to the chemical bin 108 content or acid bin 110 content to the control unit 104.

The water level sensors 120 are configured to measure the water level of the pool 102. Although two water level sensors 120 are shown in FIG. 1, in other examples, only one water level sensor 120, or more than two water level sensors 120, may alternatively be used. The water level sensors 120 are configured to wirelessly transmit the information relating to the water level in the pool 102 to the control unit 104. The water level sensors 120 are positioned on a wall of the swimming pool 102 at a height on the wall that corresponds to a desired water level to be maintained.

The control unit 104 is configured to also receive information from the water meter 124 and the ambient temperature and relative humidity sensor 126. The water meter 124 is in fluid communication with the flow of water extracted from the pool 102 by the pump 112. The water meter 124 is configured to detect the water moving through the system and wirelessly transmits this information to the control unit 104. The ambient temperature and relative humidity sensor 126 is configured to detect the temperature and humidity in the immediate environs of the swimming pool 102. The ambient temperature and relative humidity sensor 126 wirelessly transmits this information to the control unit 104.

The autonomous testing unit 150 is configured to test water drawn from the pool 102 by application of reagents. The reagents used may be those typically used in conventional manual titration testing methods. The tests conducted are standard in the industry to ensure that acceptable levels of FAC, TAC, CC, acidity and alkalinity of the water are achieved.

As is known in the art, the effectiveness of the FAC in the water depends on the pH of the water. High pH values hold back progress of the microbicidal activity of residual disinfectant in the pool. An appropriate level of chlorine in the water is important for eliminating bacteria and as it is affected by the pH, and so maintaining a relatively constant pH is important. The consistency of the pH is dependent on alkalinity. Both pH and alkalinity impact the oxidation reduction potential (ORP) value of the water. As is also known in the art, ORP is the water's level of potency to eliminate or deactivate pathogens, where a higher ORP value is associated with a greater disinfecting power.

The pump rpm sensor 140 is configured to detect the rpm of the pump 112 to determine a rate at which it is operating. As will be understood, monitoring pump rpm helps to identify if the pump is operating either within desired parameters or abnormally. In the example shown, the pump rpm sensor 140 is positioned at the pump 112. The pump rpm sensor 140 is configured to wirelessly transmit the rpm information to the control unit 104.

The current meter 142 is configured to detect the current flow at the pump 112 to determine the draw of current at the pump 112. As will be understood, detecting such current flow, such as if the sensor indicates a higher or lower current draw than is typical, helps to identify if the pump 112 is operating abnormally. The current meter 142 is positioned at the pump 112, and is configured to wirelessly transmit the current information to the control unit 104.

The vibration sensor 144 is configured to detect the vibration from the pump 122 in the immediate area to determine any abnormal operating conditions, such as excess vibration, that may be an indication of abnormal wear of the pump 122 or its components. The vibration sensor 144 is positioned at the pump 112, and is configured to wirelessly transmit the vibration information to the control unit 104.

The pressure sensor 146 is configured to measure the suction force applied by the pump 112 to the pool water to draw it from the pool 102. The pressure sensor 146 is situated in pipework at the pump 112, and is configured to wirelessly transmit the pressure information to the control unit 104.

The leak sensor 148 is configured to detect if water is escaping pipework at the pump 112 to determine the existence of a leak. The leak sensor 148 is situated at the pump 112, and is configured to wirelessly transmits the information to the control unit 104.

The chemical monitoring unit 106 monitors the levels of chemicals, such as chlorine or bromine, in the water and transmits the information to the control unit 104. The autonomous testing unit 150 tests the water in accordance with a predetermined testing schedule set by the control unit 104. The predetermined testing schedule may be at set intervals. For example, a typical testing schedule for a commercial pool includes testing the FAC, TAC, and CC 30 minutes prior to opening, and every two hours thereafter for pools until 30 minutes prior to closing. In the case of hot tubs, every one hour thereafter until 30 minutes prior to closing. The autonomous testing unit 150 tests for a variety of parameters. These include, by are not limited to, FAC, TAC, CC, acidity and alkalinity of the water based, with the results sent to the control unit 104.

The control unit 104 is configured to determine a rate at which water is lost from the pool 102. For example, a certain rate of evaporation from the pool 102 expected and can determined based on the measured ambient temperature and humidity in the surrounding environment. The control unit 104 is configured to determine, for example, that a leak is present based on the information received from the leak sensor 148.

The control unit 104 is configured to receive the information from the sensors, the chemical monitoring unit 106, and the autonomous testing unit 150. Based on the received information, the control unit 104 is configured to determine the status of the water in the pool 102 and the pool components. The information from the chemical monitoring unit 106 can be used by the control unit 104 to determine if the water has not only a safe level of chemicals present, but a level effective for sanitization of the water. The control unit 104 is configured to determine that the level of chemicals in the water is acceptable, too high, or too low.

The autonomous testing unit 150 is configured to test the water by application of reagents, and to communicate this information to the control unit 104. The control unit 104 is configured to process the information from the autonomous testing unit 150 to generate test results.

The control unit 104 is operatively coupled to the cloud computer 130 where the information from the sensors, the chemical monitoring unit 106, and the autonomous testing unit 150 is stored, and processed. The cloud computer 130 includes a machine learning component to analyse the information received from the control unit 104. The machine learning component determines, from the received information, when application of chemicals to the pool water may be required in the future to maintain the safe and effective level. This determination is displayed to the user at the end node computer 132.

The control unit 104, which is operatively coupled to the pump 112, is configured to control the pump 112 based on information received from the pump rpm sensor 140, the current meter 142, the vibration sensor 144, the pressure sensor 146, and the leak sensor 148. The control unit 104 may control the pump 112 so that it is activates or deactivates based on the information received. The control unit 104 may shorten or lengthen the interval between activations of the pump 112 based on the information received. The control unit 104 may increase or decease the intensity of operation of the pump 112 based on the information received. For example, based on information received the control unit 104 may determine that the pump 112 is required to operate more frequently, or for longer intervals.

The control unit 104 is configured to send the information from the pump rpm sensor 140, the current meter 142, the vibration sensor 144, the pressure sensor 146, and the leak sensor 148 to the cloud computer 130. The machine learning component of the cloud computer 130 is configured to determine, from the received information, when failure of components is predicted, for example when the pump 112 may have reached the end of its expected lifetime. This determination is displayed to the user at the end node computer 132. The cloud computer 130 is also configured to generate a maintenance schedule for the pool 102. The maintenance schedule is generated by the machine learning component of the cloud computer 130. The maintenance schedule is based on the information received from the control unit 104. The maintenance schedule includes, for example, how frequently the pool 102 needs to be topped up with additional water, an estimation of when the pump 112 may require servicing or replacement, and an estimation of when the chemical bin 108 and the acid bin 110 may require filling. In some cases, the maintenance schedule includes estimations of expected failure of other components in the system 100, such as the sensors, or components of filtration.

The end node computer 132 is operatively coupled to the cloud computer 130. The end node computer 132 is for example, a desktop or laptop computer, or a handheld device such as a smartphone or tablet or similar. The control unit 104 transmits the information provided by the water level sensors 120, the bin level sensors 122, the water meter 124, the ambient temperature and relative humidity sensor 126, the pump rpm sensor 140, the current meter 142, the vibration sensor 144, the pressure sensor 146, the leak sensor 148, the chemical monitoring unit 106, and the autonomous testing unit 150 to the cloud computer 130, which in turn is configured to send the information at the end node computer 132 for display and viewing by the user. This enables the user, for example a pool operator or custodian, to have access to the information regarding the water status and quality in the pool 102, as well as the to the information regarding the status of components in the pool 102.

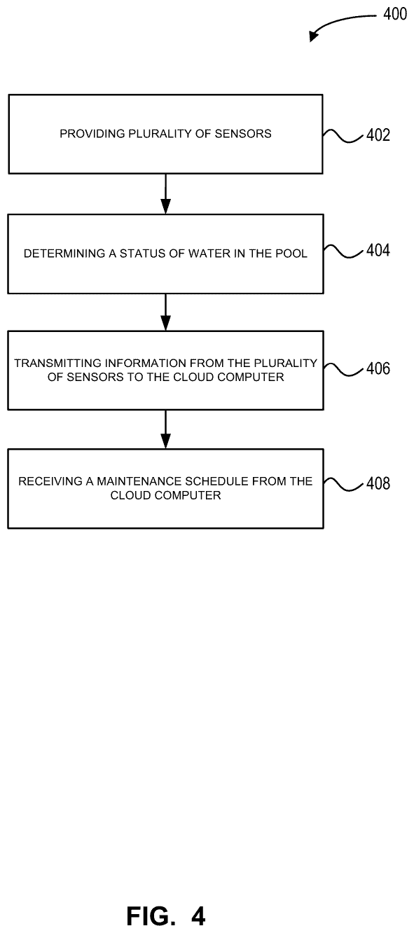

Referring now to FIG. 4 there is illustrated a method 400 for pool monitoring used by the system 100. At block 402, a plurality of sensors is provided. The sensors may include, but are not limited to, the water level sensor 120, the bin level sensor 122, the water meter 124, the ambient temperature and relative humidity sensor 126, the pump rpm sensor 140, the current meter 142, the vibration sensor 144, the pressure sensor 146, and the leak sensor 148. The sensors provide information on their respective detected parameters to the control unit 104.

At block 404, the status of the water in the pool 102 is determined. This is based on the information received by the control unit 104. The control unit 104 can determine, for example, that the water level is too low based on the information from the water level sensors 120. The control unit 104 can determine, for example, and of i) that the amount of chemical or acid in the chemical bin 108 and the acid bin 110 is low based on the information from the bin level sensors 122; ii) an abnormality in the water flow based on the information from the water meter 124; iii) fluctuations in the temperature and humidity in the immediate surroundings of the pool 102 based on the information from the ambient temperature and relative humidity sensor 126; iv) that the rpm of the pump 112 is higher or lower than typical, or that the pump 112 is or is not operating, based on the information from the pump rpm sensor 140; v) that the current draw for the pump 112 is higher or lower than typical, or that it is or is not present indicating that the pump 112 is or is not operational, based on the information from the current meter 142; vi) an abnormal level of vibration from the pump 112 which may indicate an abnormal operating condition for the pump 112, based on the information from the vibration sensor 144; and/or vii) a higher or lower level of suction force from the pump 112 than is typical based on the information from the pressure sensor 146.

At block 406, the control unit 104 transmits the information received and the determined status of the water, to the cloud computer 130. The cloud computer 130 receives the information from the control unit 104, stores it and processes it.

At block 408, the cloud computer 130 generates a maintenance schedule for the pool 102. The maintenance schedule is generated by the machine learning component of the cloud computer 130. The maintenance schedule is based on the information received from the control unit 104. The maintenance schedule includes, for example, how frequently the pool 102 needs to be topped up with additional water, an estimation of when the pump 112 may require servicing or replacement, and an estimation of when the chemical bin 108 and the acid bin 110 may require filling. In some cases, the maintenance schedule includes estimations of expected failure of other components in the system 100, such as the sensors, or components of filtration.

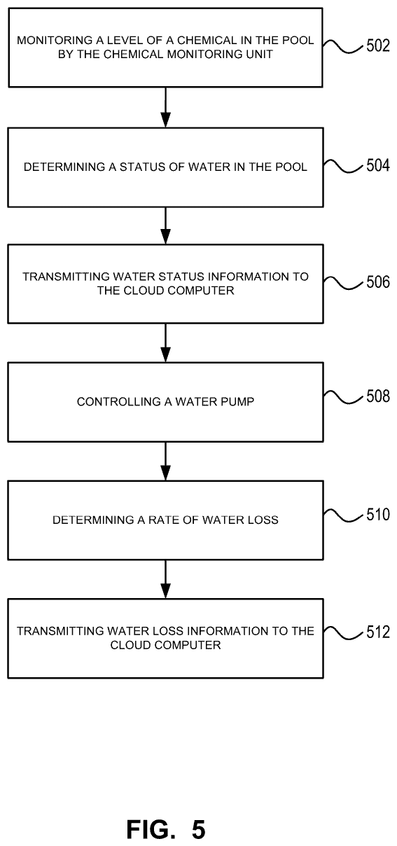

Referring now to FIG. 5, additional steps 500 for the method for pool monitoring 400 are illustrated. At block 502, the chemical monitoring unit 106 monitors a chemical level in the pool 102. The chemical level may be, for example, a level of chlorine or bromine. This information is sent to the control unit 104.

At block 504, the control unit 104 determines a status of the pool water based on the information from the chemical monitoring unit 106. This is for example, that the level of chemicals in the water is acceptable, too high, or too low.

At block 506, the information from the chemical monitoring unit 106 and the status of the pool 102 determined the by the control unit 104 is sent to the cloud computer 130. The cloud computer 130 stores and analyses the received information.

At block 508, the pump 112 is controlled by the control unit 104. Based on information received the control unit 104 may determine that the pump 112 is required to operate more frequently, or for longer intervals. This information is sent to the cloud computer 130. The cloud computer 130 stores and analyses the received information.

At block 510, the control unit 104 determines a rate of water loss in the pool 102. For example, a certain rate of evaporation from the pool 102 is expected and can be determined based on the ambient temperature of the pool 102 and the humidity in the surrounding environment. The control unit 104 may determine, for example, based on the determine rate of water loss, that a leak is present based on the information received from the leak sensor 148.

The cloud computer 130 uses the received information relating to the chemical level, pump 112 operation, and water loss to revise, where necessary, the generated maintenance schedule. Trends are identified for, for example, the frequency and volume of leaks, the water level over time, the frequency of the pump 112 operating and its duration of operation, the chemical level in the pool 102 over time, frequency of water top up required in the pool 102, the level of chemical stored in the chemical bin 108 and the acid bin 110. These trends influence the maintenance schedule and provide an estimation to the pool operator or custodian of when the pump 112 may require replacing, when the chemical bin 108 and the acid bin 110 may require refilling, the amount of chemicals to be added to the pool 102 and how frequently, the amount of water to be added to the pool 102 and how frequently, the temperature of the pool environment, how often cleaning of the pool 102 is required, and when leaks are present.

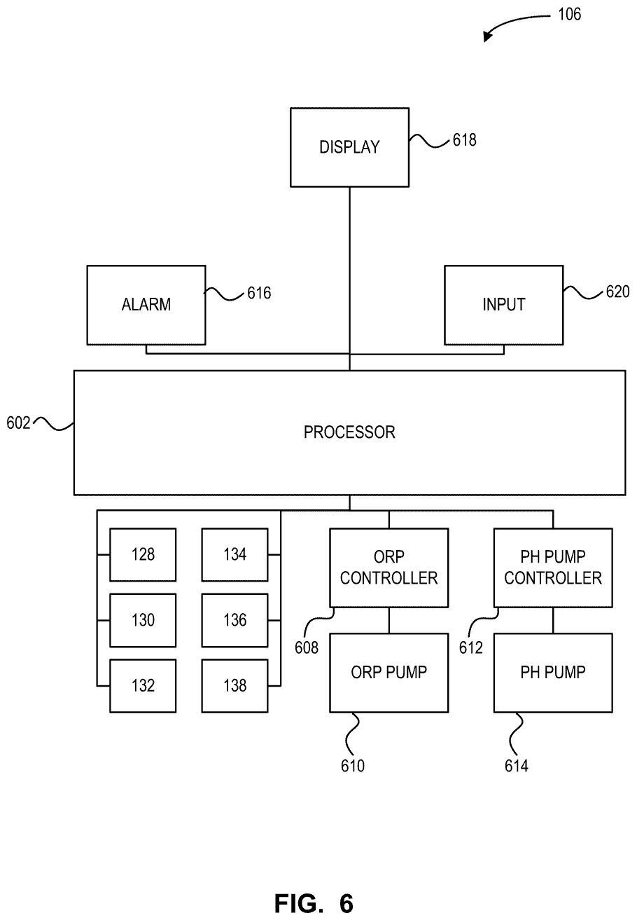

Referring now to FIG. 6, there is illustrated a schematic block diagram of an example of the chemical monitoring unit 106. The chemical monitoring unit 106 is generally configured to test the pool water to determine the status of the pool water. In the example shown, the chemical monitoring unit 106 includes a pH sensor 128, an oxidation-reduction potential (ORP) sensor 130, a water temperature sensor 132, a total dissolved solids (TDS) sensor 134, a turbidity sensor 136, a flow sensor 138, a processor 602, an ORP controller 608, an ORP pump 610, a pH pump controller 612, a pH pump 614, an alarm 616, a display 618, and an input module 620. Although in the example shown the chemical monitoring unit 106 is shown as including six (6) sensors (the pH sensor 128, the ORP sensor 130, the water temperature sensor 132, the TDS sensor 134, the turbidity sensor 136, and the flow sensor 138) other sensor configurations are possible. In some cases, the chemical monitoring unit 106 may alternatively have fewer or more sensors configured to monitor various, and possibly additional, pool parameters.

The processor 602 of the chemical monitoring unit 106 is operatively connected to the ORP controller 608, the ORP pump 610, the pH pump controller 612 and the pH pump 614. The processor 602 is also operatively connected to the sensors 128, 130, 132, 134, 136 and 138, alarm 616, display 618, and input module 620.

The sensors 128, 130, 132, 134, 136 and 138 are in fluid communication with the pool water and are positioned, for example, in pipework coupled to the pump 122. In one example, the 128, 130, 132, 134, 136 and 138 are accommodated within a housing that is positioned in fluid communication with the pool water. Each of the sensors 128, 130, 132, 134, 136 and 138 is configured to measure a respective parameter. In particular: i) the pH sensor 128 is configured to measure the potential acidity of the water in the pool 102; ii) the ORP sensor 130 is configured to measure the ORP level in the water of the pool 102; iii) the water temperature sensor 132 is configured to measure the temperature of the water in the pool 102; iv) the TDS sensor 134 is configured to measure the amount of organic and inorganic materials dissolved in the water; v) the turbidity sensor 136 is configured to measure the clarity of the water, by measuring the amount of light scattered as a result of suspended solids in the water; and vi) the flow sensor 138 is configured to measure the flow of water across the pH sensor 128, the ORP sensor 130, the water temperature sensor 132, the TDS sensor 134, and the turbidity sensor 136.

The chemical monitoring unit 106, in turn, is configured to send the information received the pH sensor 128, the ORP sensor 130, the water temperature sensor 132, the TDS sensor 134, the turbidity sensor 136, and the flow sensor 138 to the control unit 104.

The pH sensor 128 is configured to sample the water, measure acidity thereof, and generate and send information on the measured acidity of the water to the processor 602. As is known in the art, the ideal pH range for pool water is about 7.4 to 7.6. A pH level below 7.4 may result in eye irritation, corrosion of equipment, and damage to the pool surface. If the pH exceeds 7.6, the efficacy of the sanitizer is reduced and the water may become cloudy and eye irritation may occur.

The ORP sensor 130 is configured to sample the water, measure ORP thereof, and generate and send information on the measured ORP of the water to the processor 602. As is known in the art, ORP is a measure of sanitizer activity and bacteriological water quality.

The water temperature sensor 132 is configured to sample the water, measure temperature thereof, and generate and send information on the measured temperature of the pool water to the processor 602.

The TDS sensor 134 is configured to sample the water, measure a level of dissolved solids, and generate and send information on the measured dissolved solids level to the processor 602. As is known in the art, TDS naturally builds up in swimming pools, and in commercial pools using liquid chlorine, the TDS can accumulate and increase by several thousand ppm per year. In covered pools or indoor pools, which receive no rain and experience little evaporation, dilution occurs very slowly. A high TDS level can interfere with achieving desired chlorine levels and desired water clarity. A high TDS level may also cause low-grade corrosion to metal pipes, ladders, and lights in a pool, due to an adjuvant increase in the pool water conductivity.

The turbidity sensor 136 is configured to sample the water, measure the clarity thereof, and generate and send information on the measured clarity to the processor 602.

The flow sensor 138 is configured to measure the rate of flow across the multiple sensors and provides signals to the processor 602 to disable the supply of chemicals to the pool 102 when the pump 112 is off or little to no flow is detected.

The ORP controller 608 is operatively coupled to the processor 602 and to the ORP pump 610. The ORP pump 610 is configured to control the flow of chemical into the pool. The chemical monitoring unit 106 is operatively connected to the chemical bin 108 and the acid bin 110. The chemical monitoring unit 106 controls the flow of chemical and/or acid from the chemical bin 108 and/or the acid bin 110, respectively, as needed. The pH pump controller 612 is operatively coupled to the processor 602 and to the pH pump 614. The pH pump 614 controls the flow of an acid into the pool, for example, muriatic acid.

The processor 602 is configured to receive information from the sensors 128, 130, 132, 134, 136 and 138 about their respective measured parameters. The processor 602 is configured to determine, based on the received information, if the filtration system of the pool 102 is operational. If the filtration system of the pool 102 is determined to be not operational, the processor 602 is configured to signal to the ORP controller 608 to deactivate the ORP pump 610, and to signal to the pH pump controller 612 to deactivate the pH pump 614. When the processor 602 determines that the filtration system of the pool 102 is operational and water is flowing through the pool system again, the processor 602 is configured to signal to the OPR controller 608 and the pH pump controller 612 to activate the ORP pump 610 and the pH pump 614. Upon activation, the ORP pump 610 is configured to provide a flow of chemicals from the chemical bin 108 into the water, and the pH pump 614 is configured to provide a flow of acid from the acid bin 110 into the water.

The display 618 is configured to display visual output. In some cases, the display 618 may be combined with the input module 620, such as in the case of a touch-sensitive screen, in which case the display 618 is further configured to receive user input. In other cases, the input module 620 is separate from the display 618. The display 618 is configured to display the pH of the pool 102, the ORP level of the pool 102, the flow rate of the pool 102, the temperature of the water, the TDS, and turbidity of the pool 102, for viewing by a user, such as for example a pool operator or a custodian. In some cases, the display 618 displays a desired setpoint associated with each parameter. The user can therefore view the current reading relative to the desired setpoint.

A desired setpoint for the desired pH of the pool 102 and ORP level is input by the user through the input module 620. The processor 602 is configured to compare the information received from the sensors 128, 130, 132, 134, 136 and 138 to the desired setpoints, and determine the required action in order to achieve the desired setpoints. The required action may involve, for example, increasing or decreasing flow from one or both of the ORP pump 610 and the pH pump 614, or deactivating or activating one or both of the ORP pump 610 and the pH pump 614. Desired setpoints for other parameters can be input by the user through the input module 620 in a similar manner.

An alarm 616 is operatively coupled to the processor 602. The processor 602 is configured to activate the alarm 616 in the event one or more of the detected parameters deviates from the respective setpoints, to alert the pool operator or custodian that the processor 602 has detected a deviation from at least one setpoint. The alarm 616 includes an audible signal and/or a visual indicator on the housing or the display 618. The alarm 616 may be triggered, for example, under the following conditions: i) if the pH is greater than a setpoint or less than another setpoint, for example greater than 8.0 and less than 7.0; ii) if the ORP level is greater than a setpoint or less than another setpoint, for example greater than 900 and less than 500; and/or iii) if the flow sensor 138 does not detect any water flow across the sensors 128, 130, 132, 134, 136 and 138. The setpoints are configurable and are set by the pool operator or custodian at the display 618.

In some cases, the display 616 and/or the housing incorporates a plurality of light emitting diodes (not shown) operatively coupled to the processor 602 and corresponding with information from the sensors and the setpoints. For example, the processor 602 may cause a red-coloured light emitting diode (LED) to flash when an alarm 616 is triggered. The processor 602 may cause a yellow LED to flash when the alarm 616 that is triggered relates to the pH sensor 128 and setpoint. The processor 602 may cause a blue LED to flash when the alarm 616 that is triggered relates to the ORP sensor 130 and setpoint. The processor 602 may cause a green LED to flash when the alarm 616 that is triggered relates to the flow sensor 138.

The chemical monitoring unit 106 may be a standalone unit for monitoring the chemical level in the pool 102. Alternatively, the chemical monitoring unit 106 may be operatively coupled to the control unit 104. When operatively coupled to the control unit 104, the processor 602 provides the information from the sensors 128, 130, 132, 134, 136 and 138, its determinations as to the status of the water in relation to the respective setpoints, the alarm 616 status, and the status of the ORP controller 608, ORP pump 610, pH pump controller 612, and pH pump 614, to the control unit 104. The pool operator or custodian may then also view the information relating to the chemical monitoring unit 106 via the end node computer 132, with the cloud computer 130 storing and analysing the provided information.

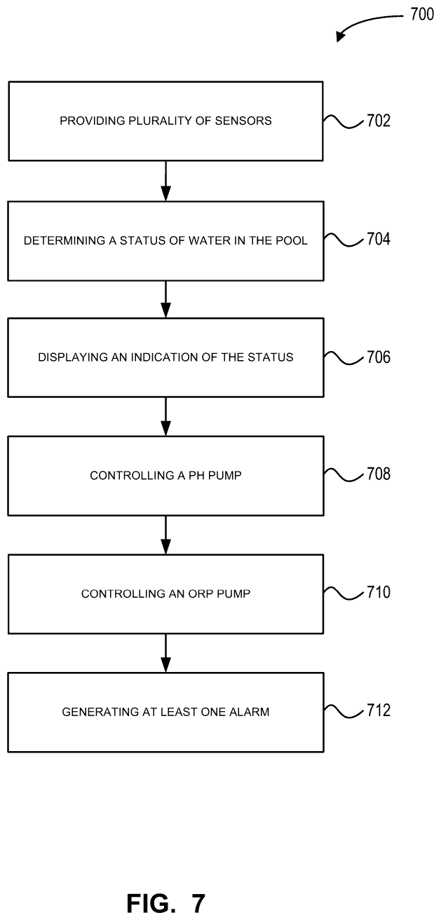

Referring now to FIG. 7, there is illustrated an example method 700 for chemical monitoring in the pool 102.

At block 702, a plurality of sensors is provided. The sensors include, but are not limited to, the pH sensor 128, the ORP sensor 130, the water temperature sensor 132, the TDS sensor 134, the turbidity sensor 136, and the flow sensor 138. The sensors 128, 130, 132, 134, 136 and 138 are in fluid communication with the pool water. The sensors 128, 130, 132, 134, 136 and 138 provide the information on their respective detected parameters to the processor 602.

At block 704, a status of the water in the pool 102 is determined. The status is determined by the processor 602 and is based on the information received from the sensors 128, 130, 132, 134, 136 and 138. In some cases, the status of the water in the pool 102 is also based on information from the ORP controller 608, and the pH pump controller 612. The status of the water may be that the pH is too high, too low, or at an adequate level. The status of the water may be that the OPR is too high, too low, or at an adequate level. The status of the water may be that there is or is not water flow at the sensors 128, 130, 132, 134, 136 and 138.

At block 706, the status of the water is displayed at the display 616. The display 618 displays to the pool operator or custodian the pH of the pool 102, the ORP level of the pool 102, the flow rate of the pool 102, the temperature of the water, the TDS, and turbidity of the pool 102. In some cases, the display 618 displays the desired setpoint associated with each parameter. The pool operator or custodian can therefore view the current reading with respect to the desired setpoint.

At block 708, the pH pump 614 is controlled. The pH pump 614 is controlled by the pH pump controller 612 which is operatively coupled to the processor 602. Based on the information received from the sensors 128, 130, 132, 134, 136 and 138, the processor 602 may determine, for example, that the pH pump 614 is to be turned on, therefore providing a supply of acid to the pool 102, or off, therefore stopping a supply of acid to the pool 102.

At block 710, the ORP pump 610 is controlled. The ORP pump 610 is controlled by the ORP controller 608 which is operatively coupled to the processor 602. Based on the information received from the sensors 128, 130, 132, 134, 136 and 138, the processor 602 may determine, for example, that the ORP pump 610 is to be turned on, therefore providing a supply of chlorine or bromine to the pool 102, or off, therefore stopping a supply of chlorine or bromine to the pool 102.

At block 712, the processor 602 generates an alarm 616. The alarm 616 is activated by the processor 602 in the event the detected parameters deviate from their respective setpoints. The alarm 616 includes the audible signal and/or visual indicator on the housing or the display 618. The alarm 616 is triggered if one or more of the detected parameters deviates from the respective setpoints. For example, the alarm 616 is triggered if the pH is greater than a setpoint or less than another setpoint, for example greater than 8.0 and less than 7.0; the alarm 616 is triggered if the ORP level is greater than a setpoint or less than another setpoint, for example greater than 900 and less than 500; and/or the alarm 616 is triggered if the flow sensor 138 does not detect any water flow across the sensors 128, 130, 132, 134, 136 and 138.

As will be understood, when operatively coupled to the control unit 104, the processor 602 provides the information from the sensors 128, 130, 132, 134, 136 and 138, its determinations as to the status of the water in relation to the setpoints, the alarm 616 status, and the status of the ORP controller 608, ORP pump 610, pH pump controller 612, and pH pump 614, to the control unit 104. The pool operator or custodian may then also view the information relating to the chemical monitoring unit 106 via the end node computer 132, with the cloud computer 130 storing and analysing the provided information.

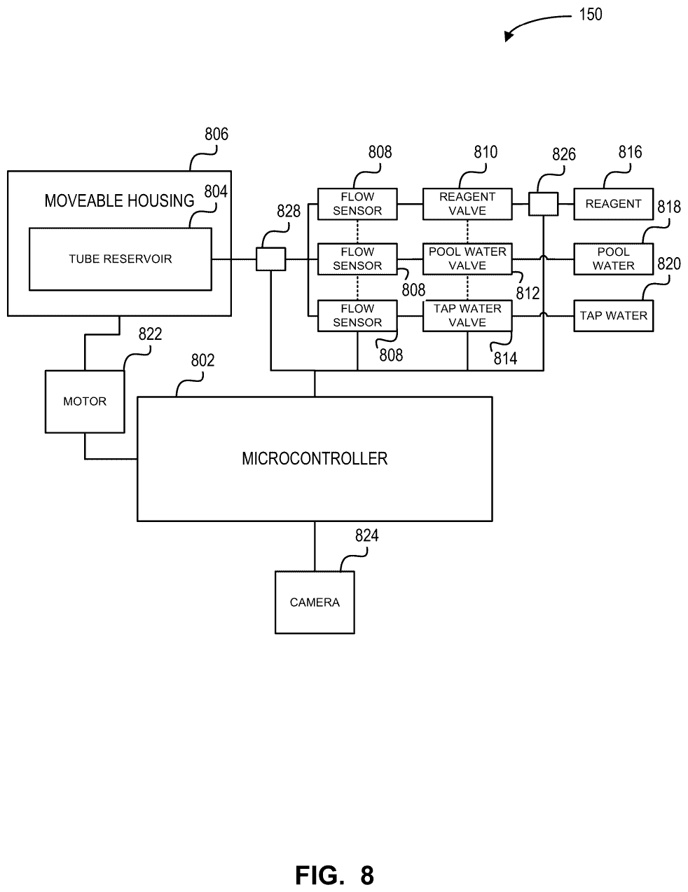

Referring to FIG. 8, there is illustrated a schematic block diagram of an exemplary apparatus for autonomous water testing in accordance with at least some embodiments.

The example autonomous testing apparatus may be the autonomous testing unit 150. The autonomous testing unit 150 includes a microcontroller 802, a sample vessel 804 positioned within a moveable housing 806, flow sensors 808, a reagent valve 810, a pool water valve 812, a tap water valve 814, a supply of reagent 816, pool water 818, and tap water 820, a motor 822, a camera 824, a pressure sensor 826, and a temperature sensor 828. The autonomous testing unit 150 can include, for example, Taylor® testing kits and reagents.

The moveable housing 806 is operatively coupled to a stepper motor 822, which is configured to facilitate movement of the sample vessel 804 situated within the moveable housing 806, upon receiving commands from the microcontroller 802. The sample vessel 804, which may for example be in the form of a tubular reservoir such as a test tube or the like, is in fluid communication with the flow sensors 808, and the valves 810, 812 and 814. The microcontroller 802 is operatively coupled to the moveable housing 806 via the motor 822, the flow meters 808, and the valves 810, 812 and 814. The camera 824 is operatively connected to the microcontroller 802.

The autonomous testing unit 150 is configured to conduct tests to determine the pH, alkalinity, TAC, CC, FAC, and total bromine in the pool water, with samples of the pool water drawn from a predetermined depth of the pool 102 from an area with a low exposure to newly added chemicals. The camera 824 is configured to capture visual test data to ensure accurate and reliable results, and provides additional context to test data. The autonomous testing unit 150 is configured to analyze and store the captured test data, to allow accurate reporting of the status of the pool water.

The autonomous testing unit 150 is configured to collect water samples from below the surface of the pool 102 according to a predetermined schedule, and accurately measure reagent to be added to the samples. As will be appreciated, the autonomous testing unit 150 advantageously provides results having improved accuracy over conventional approaches, as the results are generated by processing images captured by the camera 824, and are not based on an individual's interpretation of the image.

The reagent valve 810 is configured to control flow from the reagent supply 816, the pool water valve 812 is configured to control flow from the pool water supply 818, and the tap water valve 814 is configured to control flow from the tap water supply 820. Depending on which valve 810, 812 or 814 is opened, the sample vessel 804 is in fluid communication with at least one of the reagent supply 816, the pool water supply 818, and the tap water supply 820 at a time. The valves 810, 812 and 814 may be solenoid valves, or the like.

Although only one reagent valve 810 and reagent supply 816 is shown in FIG. 8, there may be more than one reagent valve 810 and reagent supply 816 in the autonomous testing unit 150. For example, in some embodiments, the autonomous testing unit 150 comprises a plurality of reagents, such as a bank or array of reagents, each housed in a reagent supply 816 and each associated with a respective and separately controllable reagent valve 810. The reagents contained in the reagent supply 816 may be, for example, pool reagents sold as Taylor™ reagents and manufactured by Taylor Water Technologies LLC of Maryland, U.S.A.

Each of the valves 810, 812 and 814 is connected to its respective supply 816, 818 or 820 at one end, and to a respective flow sensor 808 at its other end. The reagent supply 816 is pressurized by an air compressor (not shown), and is configured to be monitored by the microcontroller 802 via the pressure sensor 826. The sample vessel 804 is able to move below the valves 810, 812 and 814 due to the stepper motor 822 and a ball screw (not shown).

The microcontroller 802 is configured to perform the autonomous testing based on the predetermined testing schedule. During the testing, the microcontroller 802 is configured to move the sample vessel 804 along an axis to below the tap water valve 814. The sample vessel 804 and connecting pipes are washed with water from the tap water supply 820. Tubes are connected to the valves 810, 812 and 814 at the bottom to enable the ejection of the tap water. The same principle is used for filling the sample vessel 804 with a sample of pool water from the pool water supply 818 from a pre-defined depth of the pool 102. As will be understood, these movements of the sample vessel 804 are robotic in manner and are controlled by the microcontroller 802. The microcontroller 802 is configured to then move the sample vessel 804 towards the reagent supply 816, and subsequently calculate the specific amount of reagent required with the equation:

$${Q}_{b} = ( {Q}_{f} ) ( \frac{{P}_{f}}{{P}_{b}} ) ( \frac{{T}_{b}}{{T}_{f}} ) ( \frac{{Z}_{b}}{{Z}_{f}} )$$

where: Q_{b} is the volumetric flow rate at base conditions, Q_{f} is the volumetric flow rate at flowing conditions, P, T, and Z quotients are a pressure multiplier, a temperature multiplier, and a compressibility multiplier, respectively. Here, the pressure multiplier is Pf/Pb, where: Pf=pf+Pa (in psia); pf is the static gauge pressure, in psig; Pa is the atmospheric pressure, in psia; and Pb is the base pressure, in psia. The temperature multiplier is Tb/Tf, where Tb is the base temperature, ° R; Tf is the flowing temperature, ° R; and the absolute flowing temperature is ° R=° F.+459.76°. The compressibility multiplier is Zb/Zf, where Zb is the compressibility at base conditions; and Zf is the compressibility at flowing conditions.

The compressibility multiplier can be evaluated from the super compressibility factor, Fpv with the equation:

$$\frac{{Z}_{b}}{{Z}_{f}} = {( {F}_{pv} )}^{2}$$

The pressure sensor 826 is configured to measure the pressure of the liquid and send the measured value to the microcontroller 802. The temperature sensor 828 is configured to measure the temperature and send the measured value to the microcontroller 802. As the surface area of each of the valves 810, 812 and 814 is constant and pre-known, and as each reagent is constant, the microcontroller 802 is able to calculate the corresponding flow rate for the reagent when the reagent valve 810 is opened.

To test for FAC, total bromine, TAC, and CC, the microcontroller 802 is configured to draw 9 mm of pool water into the sample vessel 804. The pool water is drawn from the pool water supply 818, with the flow controlled by the pool water valve 812 and the respective flow sensor 808. The microcontroller 802 is configured to add five drops of a first reagent (e.g., Taylor® Reagent 1) and five drops of a second reagent (e.g., Taylor® Reagent 2) to the sample vessel 804. The first and second reagents are drawn from the reagent supply 816, with the flow controlled by the reagent valve 810 and respective flow sensor 808. The microcontroller 802 is then configured to agitate the sample vessel to mix the first and second reagents with the pool water. The microcontroller 802 is configured to capture an image of the sample vessel using the camera 824, and then processes the image to determine the FAC and total bromine concentration values or ranges.

To determine the TAC level, the microcontroller 802 is configured to add five drops of a third reagent (e.g., Taylor® Reagent 3) to the sample vessel 804. The microcontroller 802 is configured to capture another image of the sample vessel using the camera 824. The microcontroller 802 is configured to then draw the third reagent from the reagent supply 816, by operatively controlling by the reagent valve 810 and the respective flow sensor 808. The microcontroller 802 is configured to agitate the sample vessel to mix the reagent with the pool water that contains the first and second reagents. The microcontroller 802 processes the captured image to determine the TAC concentration range. The CC is calculated by the microcontroller 802 by subtracting the FAC value or range from the TAC value or range.

The microcontroller 802 is configured to empty the sample vessel 902 containing the pool water containing the first, second, and third reagents. The microcontroller 802 is configured to add tap water to the sample vessel 804 to clean it prior to subsequent testing. The tap water is drawn from the tap water supply 820, with the flow controlled by the tap water valve 814 and the respective flow sensor 808. The microcontroller 802 is configured to empty the tap water from the sample vessel 804 prior to the subsequent testing.

To test the pH level, the microcontroller 802 is configured to draw 44 mm of pool water into the sample vessel 804. The pool water is drawn from the pool water supply 818, with the flow controlled by the pool water valve 812 and the respective flow sensor 808. The microcontroller 802 is configured to add five drops of a fourth reagent (e.g., Taylor® Reagent 4) to the sample vessel 804. The fourth reagent is drawn from the reagent supply 816, with the flow controlled by the reagent valve 810 and respective flow sensor 808. The sample vessel is agitated to mix the fourth reagent with the pool water. After agitation, the microcontroller 802 is configured to capture an image of the sample vessel 804 using the camera 824, and then processes the image to determine the pH concentration value or range.

The pool water containing the fourth reagent is emptied from the sample vessel 804. Tap water is added to the sample vessel 804 to clean it prior to subsequent testing. The tap water is drawn from the tap water supply 820, with the flow controlled by the tap water valve 814 and the respective flow sensor 808. The tap water is emptied from the sample vessel 804 prior to the subsequent testing.

To test the alkalinity, the autonomous testing unit 150 measures 25 mm of pool water into the sample vessel 804. The pool water is drawn from the pool water supply 818, with the flow controlled by the pool water valve 812 and the respective flow sensor 808. Two drops of a fifth reagent (e.g., Taylor® Reagent 7) and 5 drops of a sixth reagent (e.g., Taylor® Reagent 7) are added to the sample vessel 804. The fifth and sixth reagents are drawn from the reagent supply 816, with the flow controlled by the reagent valve 810 and respective flow sensor 808. The sample vessel 804 is agitated to mix the fifth and sixth reagents with the pool water. The camera 824 captures another image of the sample vessel 804. The microcontroller 802 processes the captured image to determine that the pool water has changed colour to green. One drop of a seventh reagent (e.g., Taylor® Reagent 9) is added to the sample vessel 804. The seventh reagent is drawn from the reagent supply 816, with the flow controlled by the reagent valve 810 and respective flow sensor 808. The sample vessel 804 is agitated to mix the fifth and sixth reagents with the pool water. The camera 824 captures another image of the sample vessel 804. The microcontroller 802 processes the captured image to determine that the pool water has changed colour from green to red. If the pool water has not turned red, another drop of the seventh reagent is added to the sample vessel and the process is repeated until the pool water has changed colour to red.

The pool water containing the fifth, sixth, and seventh reagents is emptied from the sample vessel 804. Tap water is added to the sample vessel 804 to clean it prior to subsequent testing. The tap water is drawn from the tap water supply 820, with the flow controlled by the tap water valve 814 and the respective flow sensor 808. The tap water is emptied from the sample vessel 804 prior to the subsequent testing.

The autonomous testing unit 150 repeats the described testing in accordance with the set testing schedule.

The moveable housing 806 provides a white background against which the sample vessel 804 is photographed by the camera 824. This allows for enhanced colour accuracy when processing the captured images.

The microcontroller 802 stores image processing algorithms that analyze the captured image or images of the testing. The algorithms detect changes in the water conditions to provide accurate information regarding the status of the pool water.

The autonomous testing unit 150 can be a standalone unit for testing pool water. In some cases, the autonomous testing unit 106 can be operatively coupled to the control unit 104. When operatively coupled to the control unit 104, the testing can be performed remotely, and the microcontroller 802 provides the information from the from the testing and its determinations to the control unit 104. The information, including test results, numerical data, and processed images are transmitted wirelessly and in real time. The pool operator or custodian can then view the information relating to the chemical monitoring unit 106 via the end node computer 132, with the cloud computer 130 storing and analysing the provided information. The pool operator or custodian receives real-time feedback via the end node computer 132.

When used as a standalone unit, the autonomous testing unit 150 is suitable in both laboratory and field use. It is portable allowing for on-stie testing, to provide flexibility in various water monitoring scenarios. For example, as part of a regular maintenance schedule by a pool operator or custodian, or as part of troubleshooting by a specialist.

The information, including test results, numerical data, and processed images are used to form reports that combine the numerical data from testing with the processed images to provide a comprehensive view of the status of the pool water. The reporting is important for compliance, regulatory, and auditing purposes.

The microcontroller 802 allows for the user, the pool operator or custodian, to set alerts relating to the performed tests to ensure that the water in the pool 102 contains safe but effective levels of chemicals, and that the water will not irritate occupants. As a result, appropriate intervention can be taken should a specific test trigger an alert and therefore avoid water quality issues. The user can set the alerts via, for example, the end node computer 132. The user can set the testing schedule via, for example, the end node computer 132.

Although the autonomous testing unit 150 is described as having one sample vessel 804, other configurations are possible. In some cases, the sample vessel 804 has more than one sample vessel 804. In some cases, the autonomous testing unit 150 has three sample vessels 804 allowing for the described sequential tests (FAC/TAC/CC, acidity, and alkalinity) to be performed simultaneously.

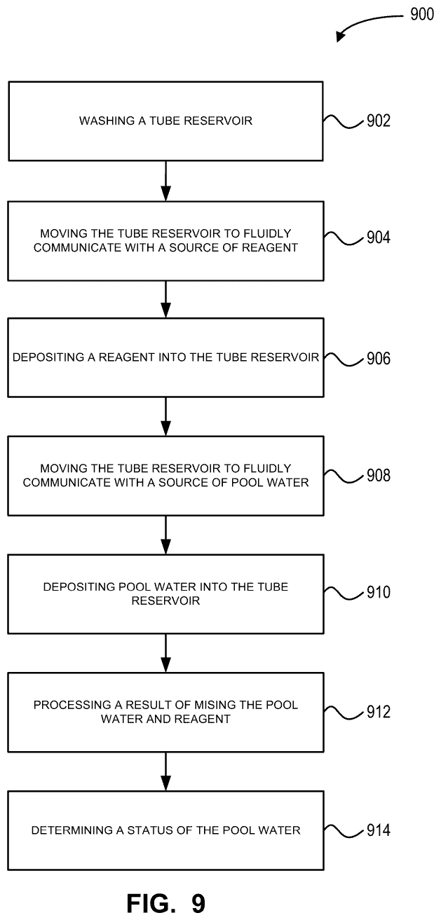

Referring to FIG. 9, there is illustrated an exemplary method 900 for autonomous water testing. Method 900 is performed by an apparatus such as the autonomous testing unit 150.

At block 902, the sample vessel 804 is washed. To achieve this, the microcontroller 802 controls the motor 822 to move the sample vessel 804 to the tap water valve 814. The microcontroller 802 operates the tap water valve 814 to open, and a flow of water from the tap water supply 820 to the sample vessel 804 is enabled.

At block 904, with the sample vessel 804 having been washed, the microcontroller 802 controls the motor 822 to move the sample vessel 804 to the reagent valve 810.

At block 906, a reagent is deposited into the sample vessel 804. The microcontroller 802 operates the regent valve 810 to open enabling a flow of reagent from the reagent supply 816 to the sample vessel 804. The flow of reagent is monitored by a flow meter 808 and the microcontroller 802 determines the desired volume of reagent required for the autonomous testing and shuts off the reagent valve 810 once the desired volume is deposited into the sample vessel 804.

At block 908, the microcontroller 802 controls the motor 822 to move the sample vessel 804 to the pool water valve 812.

At block 910, pool water is deposited into the sample vessel 804. The microcontroller 802 operates the pool water valve 812 to open enabling a flow of pool water from the pool water supply 818 to the sample vessel 804. The flow of pool water is monitored by a flow sensor 808 and the microcontroller 802 determines the desired volume of pool water required and shuts off the pool water valve 812 once the desired volume has been deposited into the sample vessel 804.

At block 912, the result of mixing the pool water with the reagent is processed. The microcontroller 802 agitates the moveable housing 806 via the motor 822 to mix the reagent with the pool water in the sample vessel 804.

At block 914, the status of the pool water is determined based on the result of mixing the pool water with the reagent. The camera 824 captures an image of the mixture which is sent to the microcontroller 802. The microcontroller 802 analyses the image to determine the status of the pool water. The analysis of the image is based on comparison of the captured result to a legend showing a variety of shades of colours for the water sample. This is used to determine the FAC, TAC, CC, acidity and alkalinity of the water based on known water titration testing principles.

The method 900 is repeated depending on the number of tests required, and the number of different reagents required to interact with the pool water to determine different parameters. Once testing is complete, the sample vessel may be washed, as in block 902.

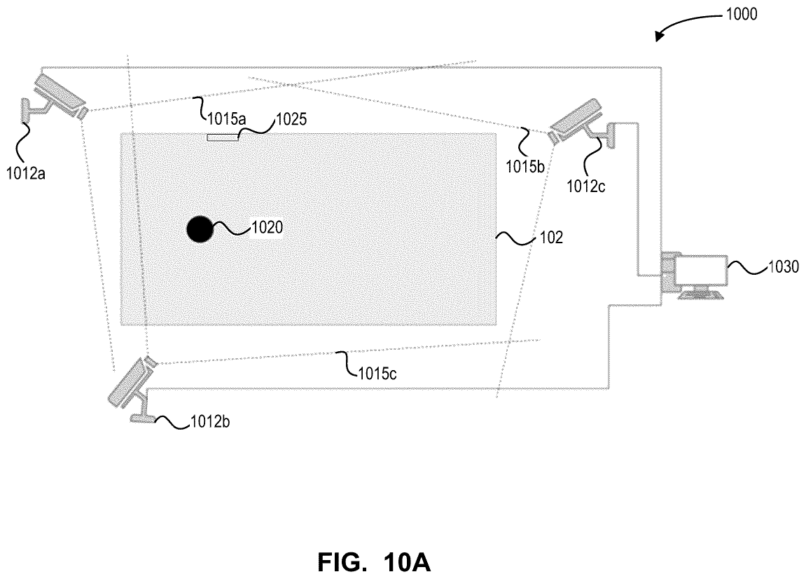

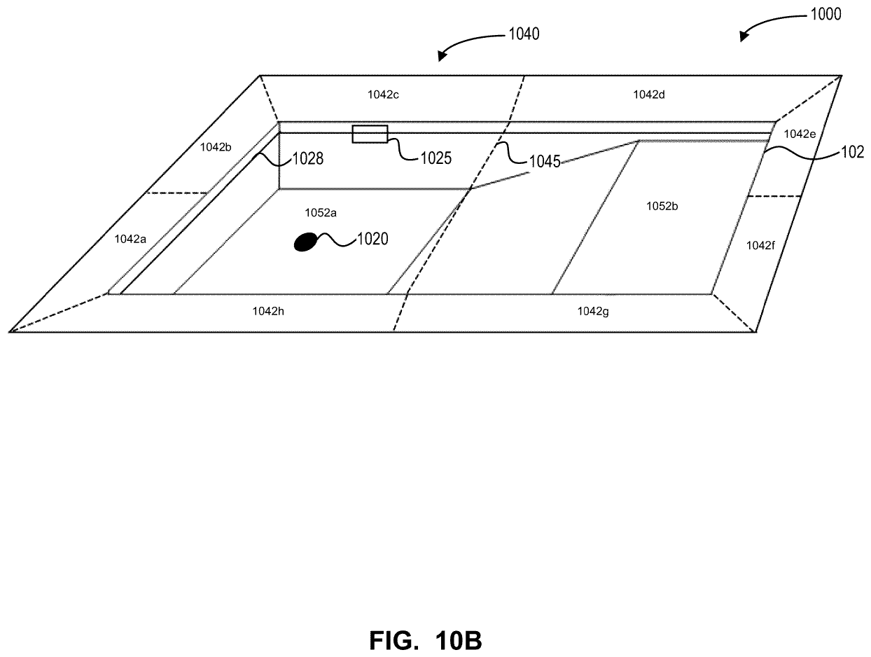

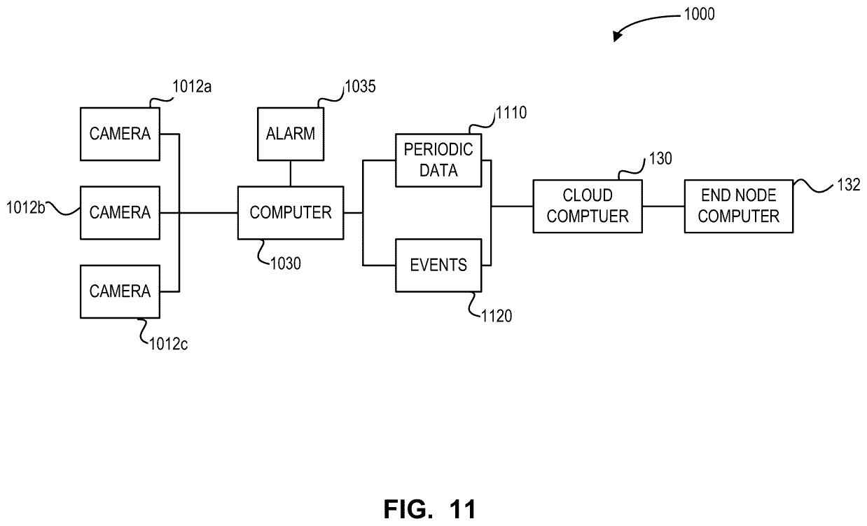

Referring now to FIGS. 10A, 10B, and 11 there is illustrated a system for pool monitoring, which is generally indicated by reference numeral 1000. System 1000 includes cameras 1012a-c, with each camera 1012a-c having a respective field of view 1015a-c of the pool, and a computer 1030. The system 1000 further includes an indicator 1020 on a bottom surface of the pool 102, and a skimmer 1025 on a wall of the pool 102. The pool 102 has a water level 1028. The cameras 1012a-c are operatively coupled to the computer 1030. The pool 102 is surrounded by an area 1040 that is divided into a plurality of deck occupancy zones 1042a-h. The pool 102 itself is divided into a plurality of water occupancy zones, namely a first water occupancy zone 1052a and a second water occupancy zone 1052b, which are separated by a boundary line 1054. The system 1000 also includes an alarm 1035 operatively coupled to the computer 1030. The computer 1030 is configured to receive images from the cameras 1012a-c and process the image data therein. The computer 1030 is operatively coupled to the cloud computer 130 and is configured to send data, including periodic data 1110 and event data 1120, to the cloud computer 130. The cloud computer 130 is configured to process the data received from the computer 1030. The pool operator or custodian can view data from the cloud computer 130 using the end node computer 132.

The system 1000 is purposed to assist the pool operator or custodian with safety and maintenance of the pool 102. The system 1000 may be incorporated into the system 100 of FIG. 1, or may be a standalone installation. The system 1000 is configured to monitor parameters such as water clarity, water level, and temperature, as well as occupancy of the pool 102 and the surrounding area 1040. The system 1000 is configured to identify human and non-human objects, as well as animals. The system 1000 identifies pre-drowning indicators and activates the alarm 1035 when pre-drowning indicators are identified.

Each of the cameras 1012a-c is positioned such that its respective field of view 1015a-c covers either the entirety of the pool 102 or a sizeable portion of the pool 102. A compete view of the pool 102 and the area 1040 surrounding the pool 102 is captured, collectively, by the cameras 1012a-c. The configuration used for the cameras 1012a-c, namely the position and angle of each of the cameras 1012a-c, depends on the size and shape of the pool 102 being monitored. The position and angle of the each of the cameras 1012a-c is adjustable in order to achieve the best overall view of the pool 102 and the area 1040 surrounding the pool 102.

Although the system 1000 is shown as comprising three cameras 1012a-c, fewer than three or more than three cameras may alternatively be used. As will be understood, at least one camera is required as a minimum with a field of view that captures the indicator 1020 and the skimmer 1025.

Preferably, at least one of the cameras 1012a-c has a full view of the indicator 1020 on the bottom of the pool 102 when the pool is empty of occupants. If the water in the pool 102 is cloudy, the indicator 1020, which in this example is a black disc, will not be is not visible to the cameras 1012a-c. This indicate that the water clarity, and hence the water quality, is poor or in other words has a value that is “low”. Low water clarity and low water quality can result in a shutdown of the pool 102, such as for maintenance to provide better water quality. By detecting low water clarity, the system 1000 informs the pool operator or custodian that action is required.

Preferably, at least one of the cameras 1012a-c has a full view of the skimmer 1025 on the wall of the pool 102. If the water level 1028 is above the skimmer 1025, the water level 1028 is too high. If the water level 1028 is below the skimmer 1025, the water level 1028 is too low.

Although the water level 1028 is determined relative to the skimmer 1025, other arrangements are possible. In some cases, another indicator on the wall of the pool 102 may be used to determine the water level 1028.

The cameras 1012a-c are configured to capture any of static images, video images, or a combination of both. The computer 1030 is configured to process the received images using computer vision algorithms to determine if the water level 1028 is adequate, and to determine if the water clarity is adequate. A maximum and a minimum acceptable water level for the pool 102 are set at the computer 1030, and are what the water level 1028 in the pool is compared against in the received images during image processing. The maximum and minimum acceptable water level correspond to, for example, the highest and lowest level of the skimmer 1025. As will be understood, the presence of occupants, floating objects, submerged objects in the pool 102 will cause the water level 1028 to fluctuate.

The cameras 1012a-c are connected to the computer 1030 by a Local Area Network (LAN) which can be wired or wireless. The computer 1030 is responsible for processing the received images from the cameras 1012a-c and extracting key information to send to the cloud computer 130. The computer 1030 serves two purposes. For privacy, the received images are processed locally in real-time at the computer 1030. They are not sent over the internet nor saved locally, and are disposed of after processing. As will be appreciated, the cost of processing data locally at the computer 1030 is much lower than the cost of using internet bandwidth to transmitting captured image data to remote processing structure.

The computer 1030 aggregates the information received from each of the cameras 1012a-c. To achieve this, the computer 1030 divides the pool 102 and the area 1040 surrounding the pool 102 into zones. As a result, data from the received images is finalized text-based data that is sent to the cloud computer 130. The finalized text-data data includes a set of keys and values in JSON format.

The computer 1030 uses a single, generic representation for all shapes and sizes of pool 102 to represent information extracted from the cameras 1012a-c. By dividing the pool 102 and the surrounding area 1040 into zones, a standard, the abstract unified representation of the pool 102 and the surrounding area 1040 is generated and is displayed at the end node computer 132. The representation shows the extracted information for all different shapes and sizes of pools and hot tubs. In this way, the pool occupancy and water condition may be displayed visualized without compromising the privacy of the bathers.

The cloud computer 130 generates the representation of the pool 102 and the area 1040 surrounding the pool 102 based on the zoning information and data from the computer 1030. The pool 102 is divided into the first water occupancy zone 1052a and the second water occupancy zone 1052b. In the example shown, the first water occupancy zone 1052a corresponds to a deep end of the pool 102, and the second water occupancy zone 1052b corresponds to a shallow end of the pool 102. For concise zone detection, ropes with contrasting colours (such as red or orange or blue/white) can be used to divide the pool 102 during a calibration phase. For example, a rope along boundary line 1054 divides the pool 102 into the first water occupancy zone 1052a and the second water occupancy zone 1052b. During calibration, the computer 1030 detects the rope 1054 in images captured by the cameras 1012a-c as the boundary between the first water occupancy zone 1052a and the second water occupancy zone 1052b. Each zone is assigned a unique identifier, such as number, and is assigned as being inside (water) or outside (the area 1040) the pool 102. The use of the unique identifier avoids confusion when the fields of view 1015a-c of the cameras 1012a-c are overlapping.

The end node computer 132 displays information extracted from the cloud computer 130 that relates to the water clarity, water level, temperature, and occupancy in the zones. A graphical representation of the pool 102 is displayed at the end node computer 132, which displays a simplified view of the pool 102 divided into the occupancy and water occupancy zones. Depending on the size on the pool 102, the pool 102 can be represented as a single water occupancy zone, or it can be divided into 2 or 4 zones. Similarly, the area 1040 surrounding the pool 102 can be divided into up to 8 occupancy zones. As will be appreciated, the graphical representation generated by the system 1000 is appropriate for any size or shape of pool or hot tub.

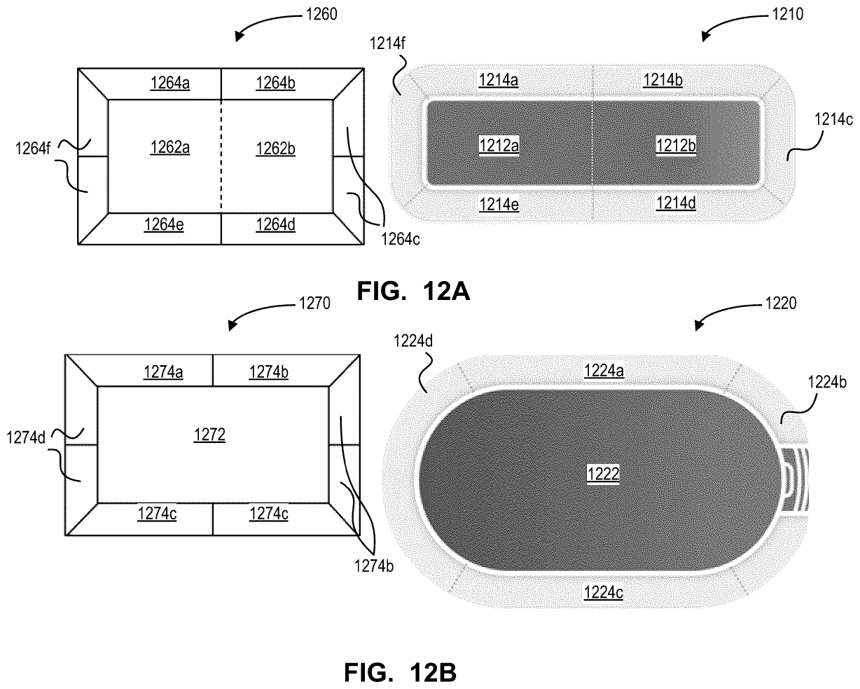

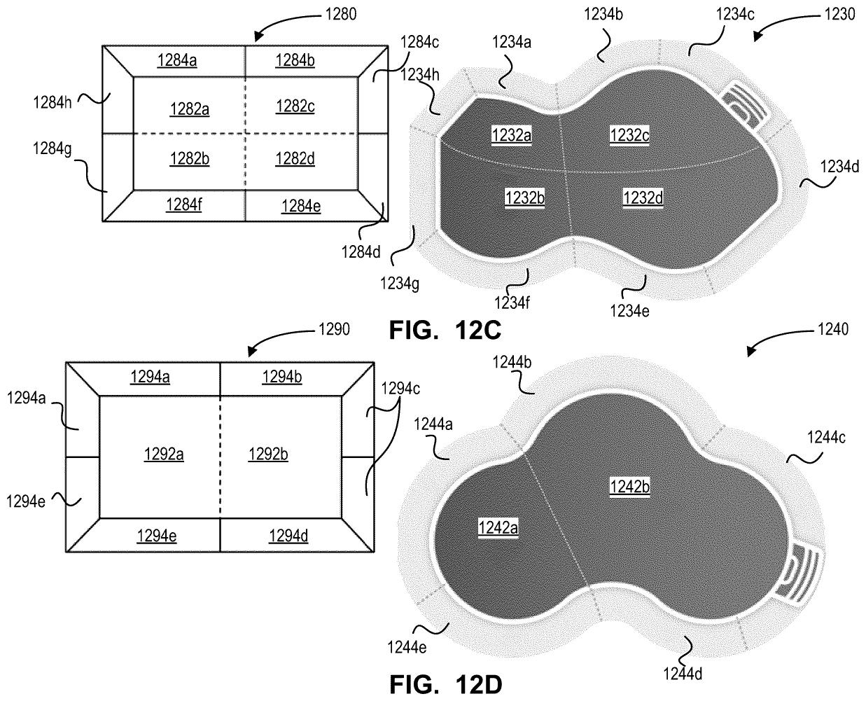

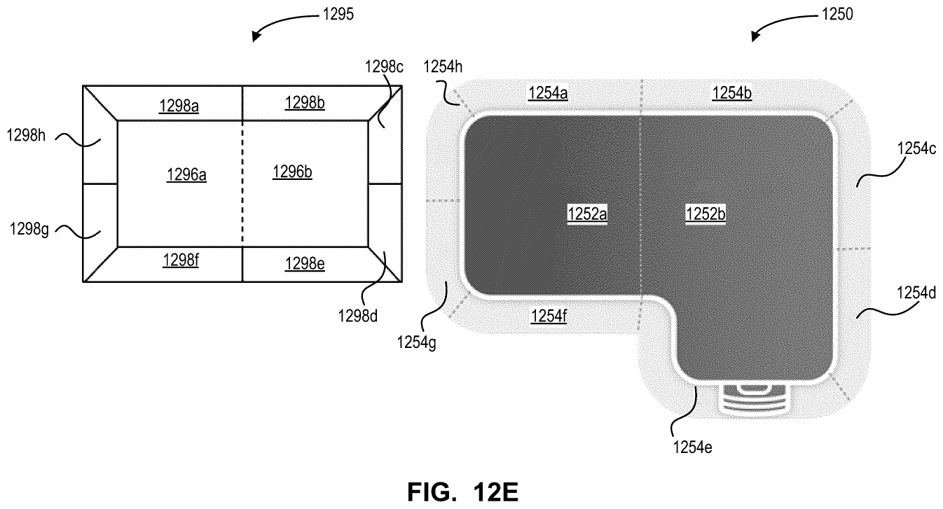

Referring now to FIGS. 12A to 12E, examples of different shapes of the pool 102, and their associated graphical representations, are shown. In FIG. 12A, an exemplary regularly shaped pool 1210, and a graphical representation 1260 corresponding to the pool 1210 generated by the system 1000, is shown. The pool 1210 is generally rectangular in shape, and is divided into two water occupancy zones 1212a, 1212b. Water occupancy zone 1212a corresponds to the deep end of the pool 1210, and water occupancy zone 1212b corresponds to the shallow end of the pool 1210. The area surrounding the perimeter of the pool 1210, which is commonly referred to as a “deck”, is divided into a plurality of deck occupancy zones 1214a-f. The graphical representation 1260 of the pool 1210 shows two virtual water occupancy zones 1262a, 1262b corresponding to the pool's 1210 water occupancy zones 1212a, 1212b. The graphical representation 1260 shows virtual deck occupancy zones 1264a-f corresponding to the pool's 1210 deck occupancy zones 1214a-f.

In FIG. 12B, another example of a regular shaped pool 1220 and a graphical representation 1270 corresponding to the pool 1220 generated by the system 1000 is shown. The pool 1220 is generally oval in shape. The pool 1220 has one water occupancy zone 1222. Around the deck of the pool 1220 is divided into a plurality of deck occupancy zones 1224a-d. The graphical representation 1270 of the pool 1220 shows one virtual water occupancy zone 1272 corresponding to the pool's 1220 water occupancy zone 1222. The graphical representation 1270 shows virtual deck occupancy zones 1274a-d corresponding to the pool's 1220 deck occupancy zones 1224a-d.

In FIG. 12C, an example of an irregular shaped pool 1230 and a graphical representation 1280 corresponding to the pool 1230 generated by the system 1000 is shown. The pool 1230 is divided into four water occupancy zones 1232a-d. Water occupancy zones 1232a and 1232b correspond to the deep end of the pool 1230, and water occupancy zones 1232c and 1232d correspond to the shallow end of the pool. The deck of the pool 1230 is divided into a plurality of deck occupancy zones 1234a-h. The graphical representation 1280 of the pool 1230 shows four virtual water occupancy zones 1282a-d corresponding to the pool's 1230 water occupancy zones 1232a-d. The graphical representation 1280 shows virtual deck occupancy zones 1284a-h corresponding to the pool's 1230 deck occupancy zones 1234a-h.

In FIG. 12D, another example of an irregular shaped pool 1240 and a graphical representation 1290 corresponding to the pool 1240 generated by the system 1000 is shown. The pool 1240 is divided into two water occupancy zones 1242a, 1242b. Water occupancy zone 1242a corresponds to the deep end of the pool 1240, and water occupancy zone 1242b corresponds to the shallow end of the pool 1240. The deck of the pool 1240 is divided into a plurality of deck occupancy zones 1244a-e. The graphical representation 1290 of the pool 1240 shows two virtual water occupancy zones 1292a, 1292b corresponding to the pool's 1240 water occupancy zones 1242a, 1242b. The graphical representation 1290 shows virtual deck occupancy zones 1294a-e corresponding to the pool's 1240 occupancy zones 1234a-e.

In FIG. 12E, another example of an irregular shaped pool 1250 and a graphical representation 1295 corresponding to the pool 1250 generated by the system 1000 is shown. The pool 1250 is divided into two water occupancy zones 1252a, 1252b. Water occupancy zone 1252a corresponds to the deep end of the pool 1250, and water occupancy zone 1252b corresponds to the shallow end of the pool 1210. The deck of the pool 1250 is divided into a plurality of deck occupancy zones 1254a-h. The graphical representation 1295 of the pool 1250 shows two virtual water occupancy zones 1296a, 1296b corresponding to the pool's 1250 water occupancy zones 1252a, 1252b. The graphical representation 1295 shows virtual deck occupancy zones 1298a-h corresponding to the pool's 1250 occupancy zones 1298a-h.

When mapping the occupancy zones and the water occupancy zones, the deep end of the pool 102 should be assigned to a water occupancy zone first. The remaining water occupancy zones are arranged relative to the deep end of the pool 102. In some cases, the floor of the pool 102 may be relatively level and without a deep end and a shallow end. In this case, the zones may be arbitrarily assigned based on length, width, for example, or the pool 102 may be assigned a single water occupancy zone.

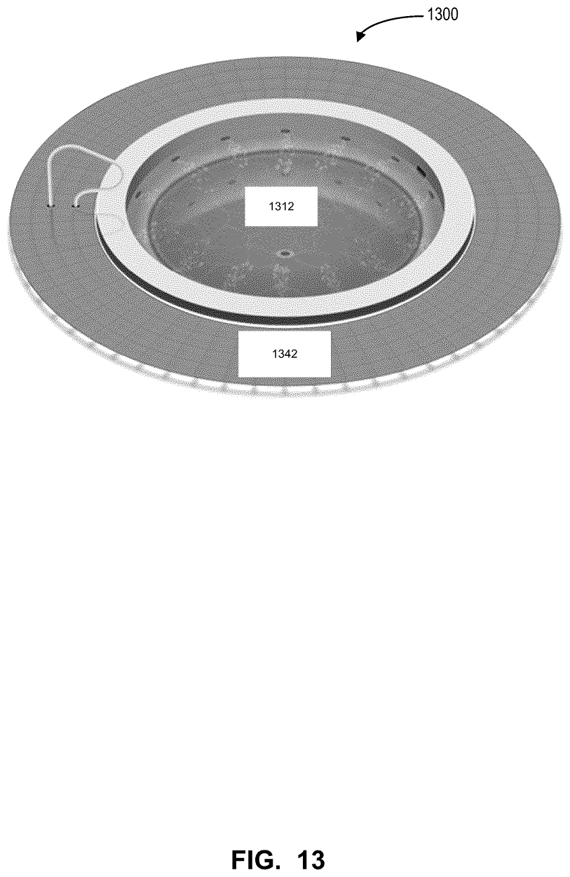

In some cases the pool 102 may be a hot tub or spa. For bodies of water that include hydro-therapy jets, such as a hot tub or spa, one water occupancy zone is assigned for the water, and one occupancy zone is assigned to the perimeter. For example, referring to FIG. 13, a graphical representation 1300 of a hot tub or spa generated by the system 1000 is shown. The graphical representation includes a virtual water occupancy zone 1312 and a virtual deck occupancy zone 1342 outside the hot tub.

As will be understood, the system 1000 shown in FIGS. 10A to 13 is purposed to collect and integrate data for autonomous monitoring of the pool 102. The computer 1030 receives the images from the cameras 1012a-c and aggregates the data to ensure that overlapping or contradictory data is integrated into the finalized text-based data, e.g., a JSON file, for sending to the cloud computer 130. The computer sends the periodic data 1110 and the event data 1120. The periodic data 1110 includes data relating to temperature, clarity, water level, and occupancy. The event data 1120 includes data relating to foreign objects present in the pool 102, as well as objects present in the pool 102 that might be drowning.

Each of the cameras 1012a-c has thermal monitoring capabilities. Each camera 1012a-c may be, for example, a thermal camera. As each camera 1012a-c has a different, although overlapping, field of view 1015a-c, each camera 1012a-c is capable of generating thermal images for different areas of the pool 102 and the surroundings. Each camera 1012a-c may have a view of all of the defined zones, or only a portion of the defined zones. The computer 1030 determines an average temperature of all points in each water occupancy zone from the images received from the cameras 1012a-c. Each camera 1012a-c sends the thermal images periodically. If the computer 1030 receives more than one thermal image for a zone within a predefined duration (e.g., 1 minute), the computer 1030 determines an average value. The time intervals between each instance of thermal images sent by the cameras 1012a-c is configurable. Preferably, a time interval of about 1 minute is used.

For determining the water clarity, each camera 1012a-c has a view of the indicator 1020 on the bottom of the pool 102. The cameras 1012a-c require calibration or a “training period” with the pool 102 being empty of occupants to adapt to the light and the distance from the indicator 1020. Once the training period is complete, the cameras 1012a-c send images of the pool 102, including the indicator 1020, to the computer 1030. The computer 1030 determines the water clarity of the pool 102 based on how visible the indicator 1020 is in the received images. Each camera 1012a-c periodically checks the view it has of the indicator 1120. The time interval between each attempt to the view indicator 1020 is, for example, about 10 minutes. The computer 1030 considers the water to be clear if it has received images from the cameras 1012a-c in the past hour where there is at least 50% visibility of the indicator 1020. The number of occupants and the effect of natural light (e.g. through windows in the immediate vicinity of the pool 102) can impact the determined water clarity level.

For determining the water level 1028 in the pool 102, the cameras 1012a-c should have a view of the skimmer 1025 on the wall of the pool 102. The cameras 1012a-c require a training or calibration period with the pool 102 to determine the size of the skimmer 1025 and the range of adequate levels. Once the training time is complete, the cameras 1012a-c send images of the skimmer 1025 and the water level 1028 with respect to the skimmer 1028. As the water level 1028 can fluctuate while the cameras 1012a-c are monitoring, the computer 1030 employs a voting mechanism for the collected water level data. If each camera 1012a-c reports that the water level is the same 1028, the computer 1030 accepts the water level 1028. If each camera 1012a-c reports that the water level is different, the computer 1030 compares the water level 1028 in the received images. If two cameras send images with a same or very similar water level, while a third camera sends an image with a different water level, the computer 1030 accepts the water level from the images with the same or similar water level.

For determining the occupancy of the pool 102 (including in each deck occupancy zone 1042a-h and water occupancy zone 1052a-b) each camera 1012a-c sends images to the computer 1030 to update the computer 1030 of any humans or other pre-trained objects that enter or leave each occupancy zone 1042a-h and each water occupancy zone 1052a-b. The computer 1030 confirms the occupancy only if all the cameras 1012a-c with a compete view of a particular zone reports the entrance or exit. Therefore, the computer 1030 will not update the occupancy of a particular zones based on images from a camera with only a partial view of that particular zone. As a result of this, the computer 1030 is always aware of the number of occupants in each zone. The other pre-trained objects include, for example, buoys, chairs, floatation aids, pool noodles, and cleaning nets.

The cameras 1012a-c are trained to detect a drowning person. The computer 1030 analyzes the received images from signs of drowning or pre-drowning indicators, e.g. a person struggling to stay above the water. If any of the cameras 1012a-c send images in which a drowning person is detected, the computer 1030 activates the alarm 1035 so that appropriate life saving action can be taken. In addition to the alarm 1035 being activated, the cameras 1012a-c record a constant stream of video for a period of time. The period of time is about 5 minutes.

The cameras 1012a-c detect any unknown objects or animals in the water occupancy zones 1052a-b. If the majority of the cameras 1012a-c with a total view of a particular water occupancy zone report the intrusion, the computer 1030 confirms the problem and activates the alarm 1035.

The temperature, water clarity, water level, and occupancy information sent to the computer 1030 is processed by the computer into the periodic data 1110 sent to the cloud computer 130. The drowning and foreign object information sent to the computer 1030 is processed by the computer into the events data 1120 sent to the cloud computer 130.

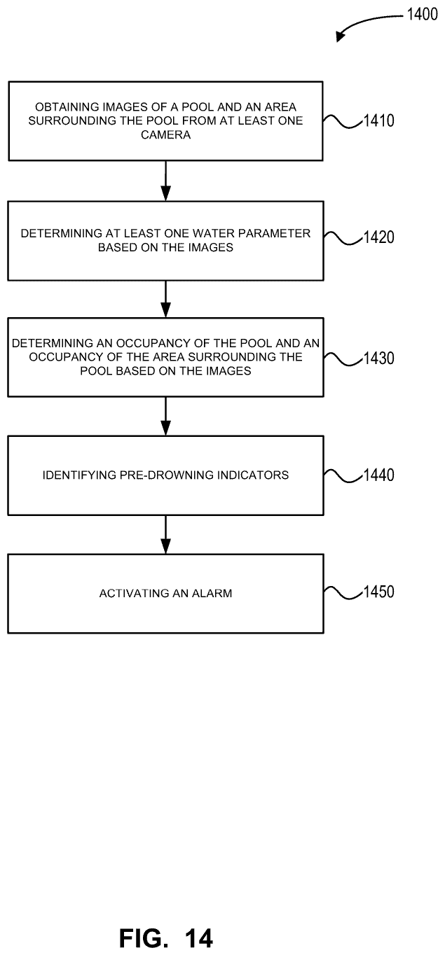

Referring now to FIG. 14, there is illustrated a method 1400 for pool monitoring. At block 1410, images of the pool 102 and the area 1040 surrounding the pool 102 are obtained from the cameras 1012a-c. The cameras 1012a-c each have their respective field of view 1015a-c. At least one of the cameras 1012a-c has a view of the indicator 1020 on the bottom of the pool and the skimmer 1025 on the wall of the pool. The images are sent to the computer 1030.

At block 1420 at least one water parameter is determined. The computer 1030 determines at least one of the water level, the water clarity, or temperature, based on the images received from the cameras 1012a-c. The water level 1028 is determined based on a comparison of the level of the water in the pool 102 with reference to the skimmer 1020. If water is above the top of the skimmer 1025, the water level 1028 is too high. If water is below the bottom of the skimmer 1025, the water level 1028 is too low. The water clarity is determined based on how visible the indicator 1020 at the bottom of the pool 102 is. At least one of the cameras 1012a-c is a thermal camera and provides thermal images for the water occupancy zones 1052a, 1052b in the pool 102.

At block 1430, the computer 1030 determines the number of occupants in the pool 102, including in each water occupancy zone 1052a, 1052b, from the images. Each camera 1012a-c sends images to the computer 1030 to update the computer 1030 of any humans or other pre-trained objects that enter or leave each deck occupancy zone 1042a-h and each water occupancy zone 1052a-b. The computer 1030 confirms the occupancy only if all the cameras 1012a-c with a compete view of a particular zone reports the entrance or exit. Therefore, the computer 1030 will not update the occupancy of a particular zones based on images from a camera with only a partial view of that particular zone. As a result of this, the computer 1030 is always aware of the number of occupants in each zone.

At block 1440, the computer 1030 determines pre-drowning identifiers. The cameras 1012a-c are trained to detect a drowning person. If any of the cameras 1012a-c send images in which a drowning person is detected, the computer 1030 activates the alarm 1035 at block 1450 so that appropriate life saving action can be taken. In addition to the alarm 1035 being activated, the cameras 1012a-c record a constant stream of video for a period of time. The period of time is about 5 minutes.

The cameras 1012a-c detect any unknown objects or animals in the water occupancy zones 1052a-b. If the majority of the cameras 1012a-c with a total view of a particular water occupancy zone report the intrusion, the computer 1030 confirms the problem and activates the alarm 1035.

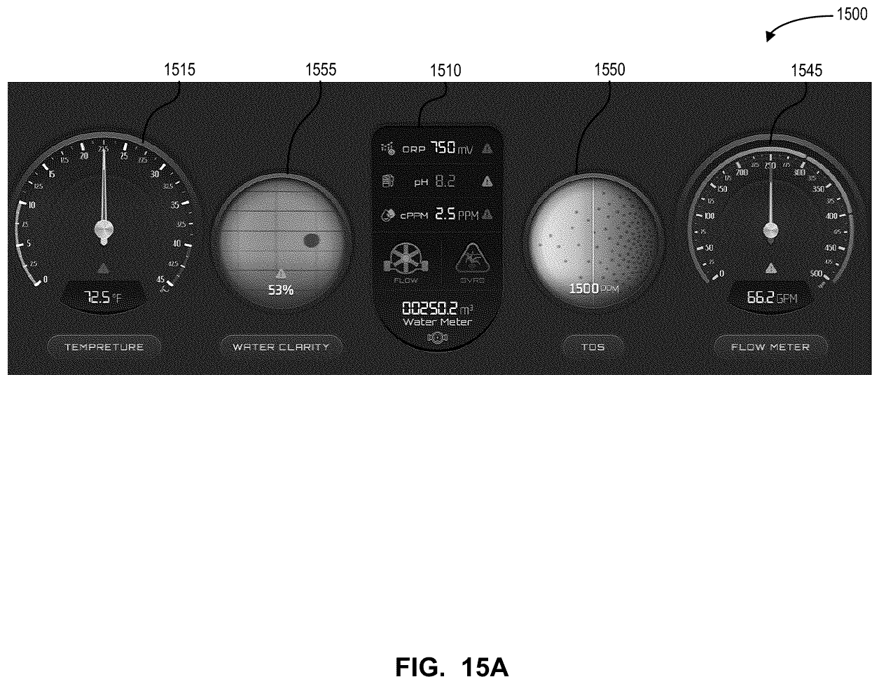

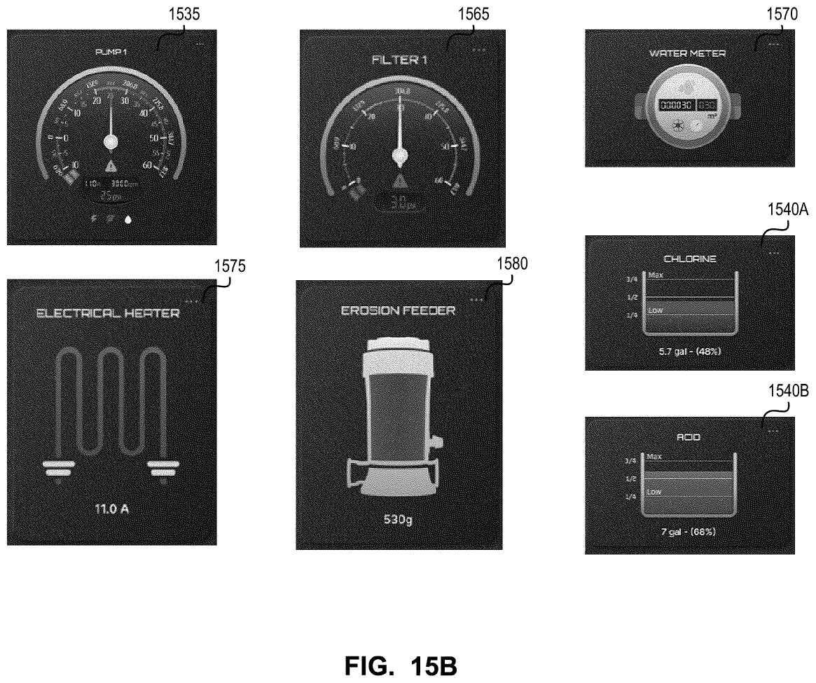

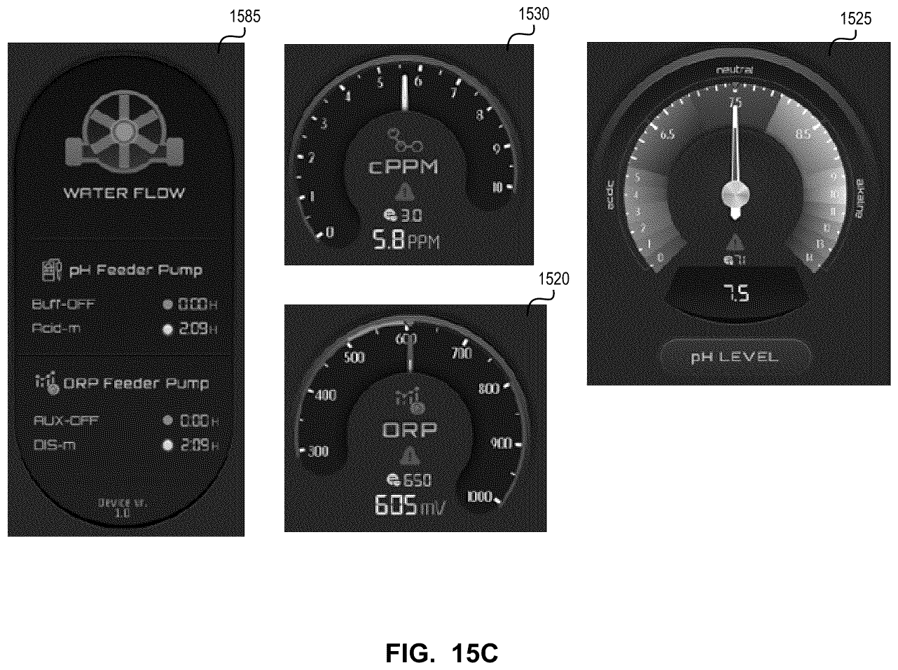

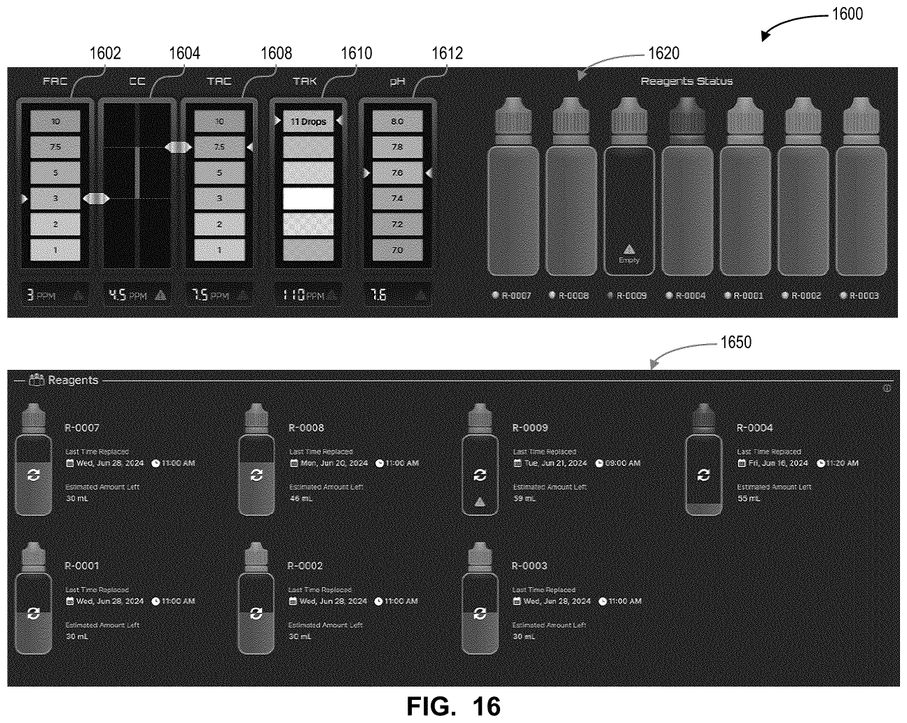

Referring to FIG. 15A, there is illustrated an example interface 1500 for displaying the information received from the control unit 104 and the chemical monitoring unit 106. The interface 1500 may display the information via dials, gauges, tables, graphs, charts, or similar.

Referring to FIG. 15A, FIGS. 15B, and 15C, there are illustrated example gauges and dials for display at the interface 1500. When the pool operator or custodian views the data relating to the water status and pool component status at the end node computer 132, the interface 1500 is provided, which includes several dials and gauges relating to parameters detected by the control unit 104 and the chemical monitoring unit 106.

The interface 1500 comprises several dials and/or gauges that digitally represent the data collected from the control unit 104 and the chemical monitoring system 106. The interface 1500 may include a key reading gauge 1510, a temperature gauge 1515, an ORP gauge 1520, a pH gauge 1525, a calculated PPM (cPPM) gauge 1530, at least one pump dial 1535, at least one chemical gauge 1540A, 1540B, a flow meter dial 1545, a TDS gauge 1550, a water clarity gauge 1555, a filter dial 1565, a water meter 1570, an electric heater gauge 1575, an erosion feeder gauge 1580, and a water summary gauge 1585. The interface 1500 may display a subset of the gauges and dials, all of them, or include other gauges for other measured or detected parameters. The interface 1500 may show the gauges and dials on one display, allowing the pool operator or custodian to view all the parameters side-by-side, and giving a whole view of the pool's 102 operation. The interface 1500 may show only some of the gauges and dials, or may display all the gauges and dials over more than one page. The dials and gauges on the interface 1500 update approximately every 20 seconds, based on the information from the sensors. The rate at which the dials and gauges update may be faster or slower.

The key reading gauge 1510 provides a reading for four key parameters: ORP level, cPPM, pH, and the flow of water. This provides an overview of the pool's 102 water status in one dial. The key reading gauge 1510 may show all of these parameters, or only one of these parameters, or other parameters deemed desirable to have in the key reading gauge 1510. The key reading gauge 1510 displays the information from the pH sensor 128, the ORP sensor 130, the temperature sensor 132, and the flow sensor 138.

The temperature gauge 1515 provides a digital number display of the water temperature and a dial gauge. The temperature gauge 1515 provides a more in-depth digital view than the temperature displayed on the key reading gauge 1510. The temperature gauge 1515 displays the information from the temperature sensor 132.

The ORP gauge 1520 provides a digital number display of the ORP level in the pool 102 and a vertical number gauge. The ORP gauge 1520 provides a more in-depth digital view than the ORP displayed on the key reading gauge 1510. The ORP gauge 1520 displays the information from the ORP sensor 130.

The pH gauge 1525 provides a digital number display of the pH level in the pool 102 and a dial gauge. The pH gauge 1525 provides a more in-depth digital view than the pH displayed on the key reading gauge 1510. The pH gauge 1525 displays the information from the pH sensor 128.

The cPPM gauge 1530 provides a digital number display of the cPPM in the pool 102 and a vertical number gauge. The cPPM gauge 1530 provides a more in-depth digital view than the cPPM displayed on the key reading gauge 1510. The cPPM gauge 1530 is determined based on the pH and ORP of the pool 102.

The at least one pump gauge 1535 provides a digital number display of, for example, the pump's 112 current draw, rpm, and pressure and a dial gauge for the pressure. The at least one pump gauge 1535 displays the information from the pump rpm sensor 140, the current meter 142, and the pressure sensor 146. In some cases, the pool 102 has more than one pump, in which case more than one pump gauge 1535 is required.

The at least one chemical gauge 1540A, 1540B provides a digital display of a chemical stored in either the chemical bin 108 or the acid bin 110. The chemical gauge 1540A shows the chlorine stored in the chemical bin 108. The chemical gauge 1540A displays information from the bin level sensor 122 about the amount of chlorine. The chemical gauge 1540B displays information from the bin level sensor 122 about the amount of acid available in the acid bin 110. Similarly, the chemical gauge 1540A may show a level of bromine, for example, should the chemical bin 108 contain bromine.

The flow meter dial 1545 provides a digital number display of the water flow and a dial gauge. The flow meter dial 1545 displays the information from the flow sensor 146.

The TDS gauge 1550 provides a visual representation of the TDS in the pool 102 and a number for the TDS in ppm. The visual representation illustrates the TDS ppm in the pool 102 with an image that includes particles in the water. A higher TDS will show more particles in the water than a low TDS. The TDS gauge 1550 displays the information from the TDS sensor 134.

The water clarity dial 1555 provides a visual representation of the actual level of clarity in the pool 102. The data for the water clarity dial 1555 is gathered from turbidity sensor 136 that is pointed to the indicator 1020 on the bottom surface of the pool 102. The indicator 1020 may have any other shape. The turbidity sensor 136 determines how clearly the indicator 1020 can be seen from the surface of the water, and a percentage value is displayed on the water clarity dial 1555. The water clarity dial 1555 also displays how visible the indicator 1020 is and the stated percentage. A percentage of 100% shows that the indicator 1020 is completely visible and the water in the pool 102 is clear. A percentage that is close to 1% shows that the water in the pool 102 is cloudy and visibility of the indicator 1020 is minimal.

The filter gauge 1565 provides a digital number display of the pressure in the filtration system and dial gauge. A pressure sensor may be positioned in the filtration system to detect the pressure. The filter gauge 1565 displays the pressure information.

The water meter 1570 provides a digital view of a totalizer in the pool 102. The totalizer measures the volume of potable makeup water or “tap” water for the pool 102. The water meter 1570 provides information on the volume of tap water for the pool 102 visually. Determining the tap water per bather is a regulatory requirement in various states or provinces in the US and Canada. The amount of tap water recommended to be added to the pool is 15 L/bather per day. The system further determines the water loss through evaporation, splash or leaks with the occupancy monitoring for the number of bathers with respect to the amount of water added.

The electric heater gauge 1575 provides a digital display of an electric water heater (not shown) used with the pool 102. The electric heater gauge 1575 shows the current drawn by the electric water heater.

The erosion feeder gauge 1580 shows, a status of a chemical block in the pool 102. In some cases, a smaller pool may utilize a chemical block, such as a chlorine puck, to maintain the chemical balance in the pool 102. The erosion feeder provides a slow release of chemical over time as the chemical block dissolves. The erosion feeder gauge 1580 provides a digital display of a status of the erosion feeder and chemical block.

The water summary gauge 1585 provides a summary view for the pH pump 614 and the ORP pump 610. The water summary gauge 1085 shows a state of the pH pump 614 and the ORP pump 610 which can be ON or OFF, and a clock count down to turn ON or OFF.

Referring now to FIG. 16, there is illustrated an example of a graphical user interface 1600 for presenting the information received from the autonomous testing unit 150. The 1600 may be displayed, for example, by the end node computer 132. The interface 1600 may display the information via dials, gauges, tables, graphs, charts, or similar.

The interface 1600 includes, for example, an FAC gauge 1602, a CC gauge 1604, a TAC gauge 1608, a TAK gauge 1610, a pH gauge 1612 and several indicators 1620 to represent each of the reagents used when testing the water.

The FAC gauge 1602 indicates the current FAC in the pool 102. The FAC gauge 1602 provides the information from the autonomous testing unit 150 relating to the test conducted to determine the FAC.

The CC gauge 1604 indicates the current CC in the pool 102. The CC gauge 1604 provides the information from the autonomous testing unit 150 relating to the test conducted to determine the CC.

The TAC gauge 1606 indicates the current TAC in the pool 102. The TAC gauge 1606 provides the information from the autonomous testing unit 150 relating to the test conducted to determine the FAC.

The TAK gauge 1608 indicates the total alkalinity of the pool 102. The total alkalinity is determined by the autonomous testing unit 150. Total alkalinity can be determined from the total number of drops of the seventh reagent (Taylor Reagent 9). The number of drops of the seventh reagent multiplied by 10 equals the concentration of alkalinity in ppm or mg/L.

The pH gauge 1612 indicates the pH of the pool 102. The pH gauge 1612 provides the information from the autonomous testing unit 150 relating to the test conducted to determine the pH.

The indicators 1620 represent each different reagent used in the testing process carried out by the autonomous testing unit 150. The interface 1600 shows 7 different indicators, each corresponding to one of the reagents used. The indicators 1620 show a discrete indication of each reagent, namely if the reagent is available or requires replenishing. The interface 1600 comprises an additional interface page 1650, which shows further information for each of the reagents. The page 1650 provides information relating to the last time each reagent was used, and an estimate of the amount of reagent remaining. As will be understood, the information shown in the interface, including in the page 1650, is provided by the autonomous testing unit 150.

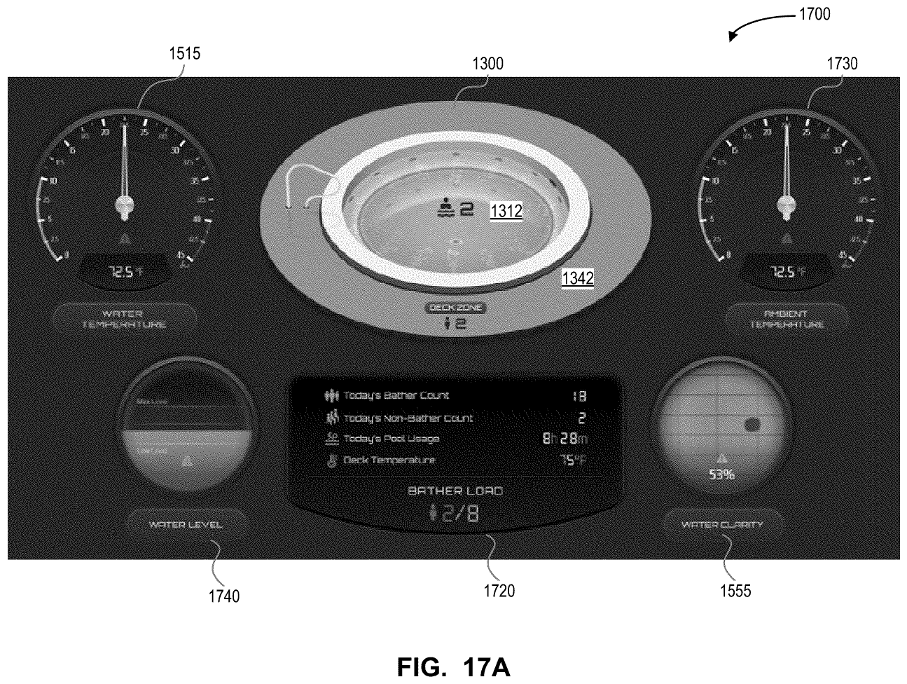

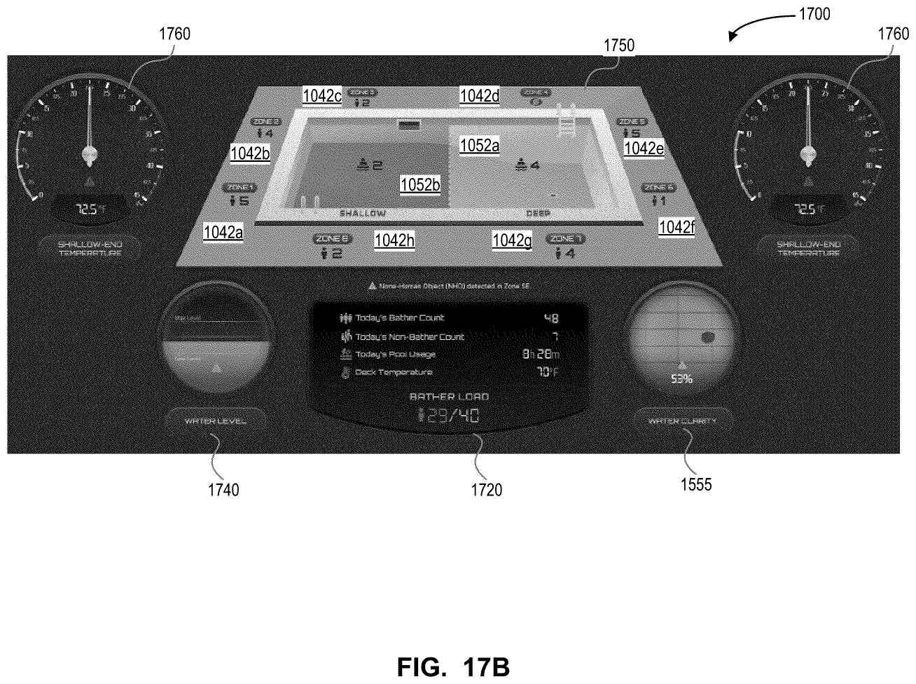

Referring now to FIG. 17A and FIG. 17B, exemplary monitoring and display graphical user interfaces for a hot tub and for a pool are shown. FIG. 17A shows an example of a hot tub interface 1700 is provided. The interface 1700 includes a summary gauge 1720, the temperature dial 1015, an ambient temperature gauge 1730, a water level gauge 1740, and the water clarity gauge 1055.

The interface 1700 also includes the graphical representation 1300 of the hot tub. The graphical representation 1300 includes the water occupancy zone 1312 and the deck occupancy zone 1342. The interface 1700 indicates the current occupancy for each of the water occupancy zone 1312 and the deck occupancy zone 1342 on the graphical representation 1300. The occupancy information is obtained from the system 1000. The computer 1030 receives images from the cameras 1012a-c from which the computer 1030 determines the number of bathers in the water occupancy zone 1312 and the deck occupancy zone 1342.

The summary gauge 1720 provides a summary of the conditions in the hot tub including the current occupancy, for bathers (namely, the number of occupants in the water occupancy zone 1312) and non-bathers (namely, the number of occupants in the deck occupancy zone 1342). The summary gauge 1720 further includes an indication of the operating time of the hot tub (namely, a time duration), and an indication of the temperature of the deck occupancy zone 1342. In some cases, the summary gauge 1720 can show other information, such as an indication from the other dials and/or gauges on the interface 1700, such as temperature, clarity, water level.

The ambient temperature gauge 1730 shows the temperature in the deck occupancy zone 1342. This information is based on image data captured by the cameras 1012a-c, where at least one of the cameras 1012a-c is a thermal camera. The computer 1030 analyzes thermal images received from the cameras 1012a-c to determine the temperature in each of the deck occupancy zones 1042a-h.

The water temperature gauge 1515 shows the temperature of the water occupancy zone 1412. This information is based on image data captured by the cameras 1012a-c, where at least one of the cameras 1012a-c is a thermal camera. The computer 1030 analyzes thermal images received from the cameras 1012a-c to determine the temperature in each of the water occupancy zones 1052a-b.

The water level gauge 1740 indicates the water level of the hot tub. This information is obtained from the system 1000 and is based on image data captured by the cameras 1012a-c, whereby the cameras 1012a-c capture images of the water with reference to the skimmer 1025, and water level information is generated by the computer 1030 and provided to the interface 1700.

The water clarity gauge 1555 indicates the clarity of the water of the hot tub. This information is obtained from the system 1000 and is based on image data captured by the cameras 1012a-c, whereby at least one of the cameras 1012a-c has a view of the indicator 1020 in the hot tub and captures images of the water. The system 1000 determines the clarity of the water based the degree of visibility of the indicator 1020, as described above, and provides the information to the interface 1700.

In some cases, the interface 1700 presents a graphical representation 1750 of the pool 102, similar to those shown in FIGS. 13A-E. Referring now to FIG. 17B, the interface includes the graphical representation 1750 includes the first water occupancy zone 1052a and the second water occupancy zone 1052b and the plurality of deck occupancy zones 1042a-h in the area 1040 surrounding the pool 102. The remainder of the interface is identical to that for the hot tub shown in FIG. 17A.

As will be understood, the various graphical user interfaces described in this and in the above embodiments can be combined and displayed as a single interface or can be displayed as separate interfaces. For example, interface 1600 and interface 1700 can be incorporated into interface 1500. In some cases, interface 1600 is a standalone interface. In some cases, interface 1700 is a standalone interface.

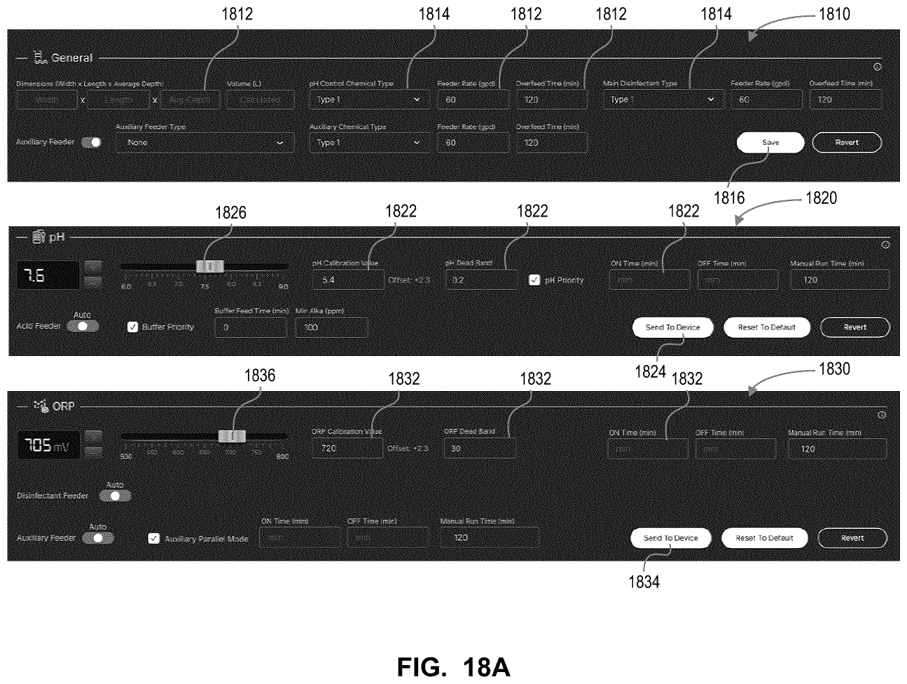

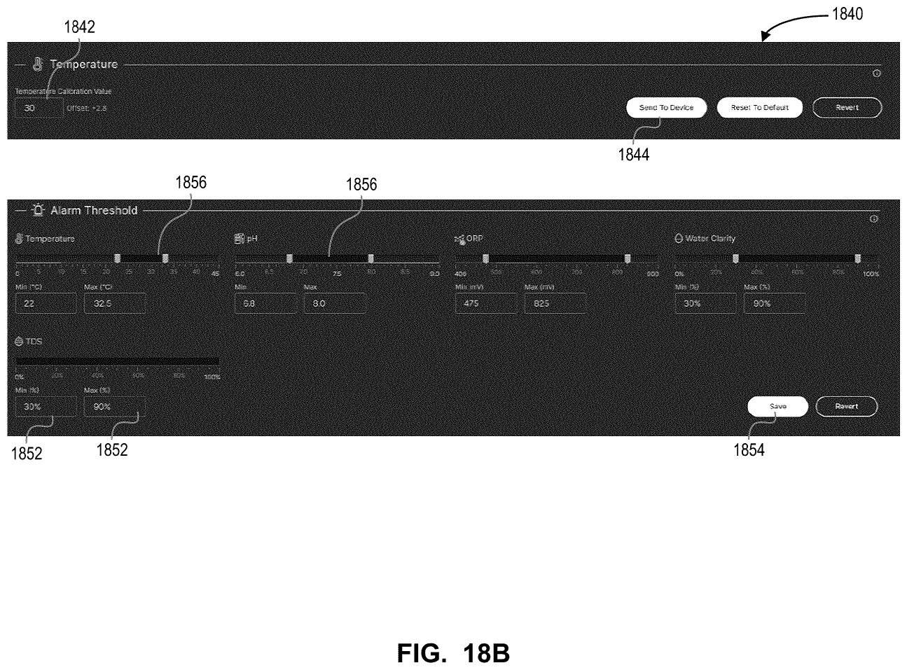

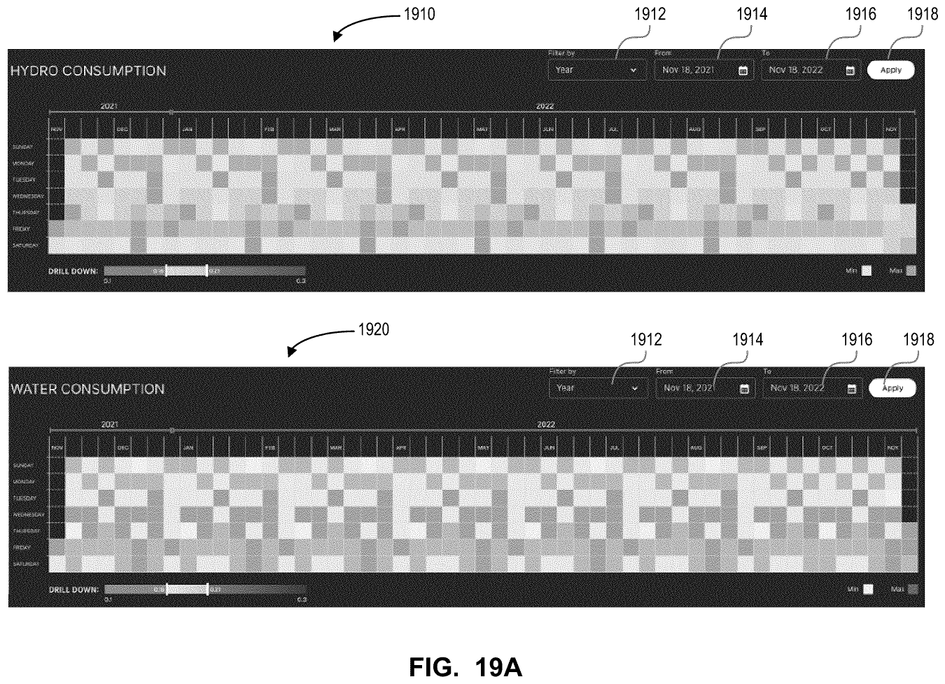

Referring now to FIGS. 18A and 18B, examples of settings pages displayed by the interface 1500 are shown. The settings pages include fields for display and entry of adjustable settings, including general settings 1810, pH settings 1820, ORP settings 1830, temperature settings 1840, and alarm thresholds 1850.

The general settings 1810 include, for example, dimensions of the pool 102, the chemicals in use, feeder rates and overfeed times for the chemicals, the disinfectant being used, and the feeder rate and the overfeed time of the disinfectant. Values for the dimensions, feeder rates and overfeed times can be entered into respective entry fields 1812. Selection of the chemical is made at the relevant selection window 1814. Once the desired information has been entered, or updated, pressing “save” button 1816 saves the general settings 1810.

The pH settings 1820 include, for example, the pH setting, a pH calibration setting, pH dead band setting, ON time, OFF time, manual run time, acid feeder, buffer feed time, and minimum alkalinity, and entry windows 1822 and a “save button” 1824. Values for the pH setting, a pH calibration setting, pH dead band setting, ON time, OFF time, manual run time, acid feeder, buffer feed time, and minimum alkalinity are entered into the relevant entry window 1822. Once the desired information has been entered, or updated, pressing the “save” button 1824 saves the pH settings 1820. In some cases, the pH settings 1820 include a pH slider 1826 which can be moved by the user to select the pH setting instead of, or in conjunction with, entering the pH setting value into the corresponding entry window 1822.

The ORP settings 1830 include, for example, the ORP setting, a ORP calibration setting, ORP dead band setting, ON time, OFF time, manual run time, disinfectant feeder ON time, OFF time, and manual run time, and entry windows 1832 and a “save button” 1834. Values for the ORP setting, a ORP calibration setting, ORP dead band setting, ON time, OFF time, manual run time, disinfectant feeder ON time, OFF time, and manual run time are entered into the relevant entry window 1832. Once the desired information has been entered, or updated, pressing the “save” button 1834 saves the ORP settings 1830. In some cases, the ORP settings 1830 include an ORP slider 1836 which can be moved by the user to select the ORP setting instead of, or in conjunction with, entering the ORP setting value into the corresponding entry window 1832.

The temperature settings 1840 include, for example, a temperature calibration value, an entry window 1842, and a “save” button 1844. The temperature calibration value is entered in the entry window 1842. Once the desired temperature calibration value has been entered, or updated, pressing the “save” button 1844 saves the temperature calibration value.

The alarm threshold settings 1850 include, for example, a minimum temperature, a maximum temperature, a minimum pH, a maximum pH, a minimum ORP, a maximum ORP, a minimum water clarity, a maximum clarity, a minimum TDS, and a maximum TDS, entry windows 1852, and a “save button” 1854. Values for the minimum temperature, maximum temperature, minimum pH, maximum pH, minimum ORP, maximum ORP, minimum water clarity, maximum clarity, minimum TDS, and maximum TDS are entered into the relevant entry window 1852. Once the desired information has been entered, or updated, pressing the “save” button 1854 saved the alarm threshold settings 1850. In some cases, the alarm thresholds 1850 include sliders 1856 which can be moved by the user to select the maximum and minimum values for the temperature alarm, the pH alarm, the ORP alarm, and the water clarity alarm instead of, or in conjunction with, entering these values into the corresponding entry windows 1852.

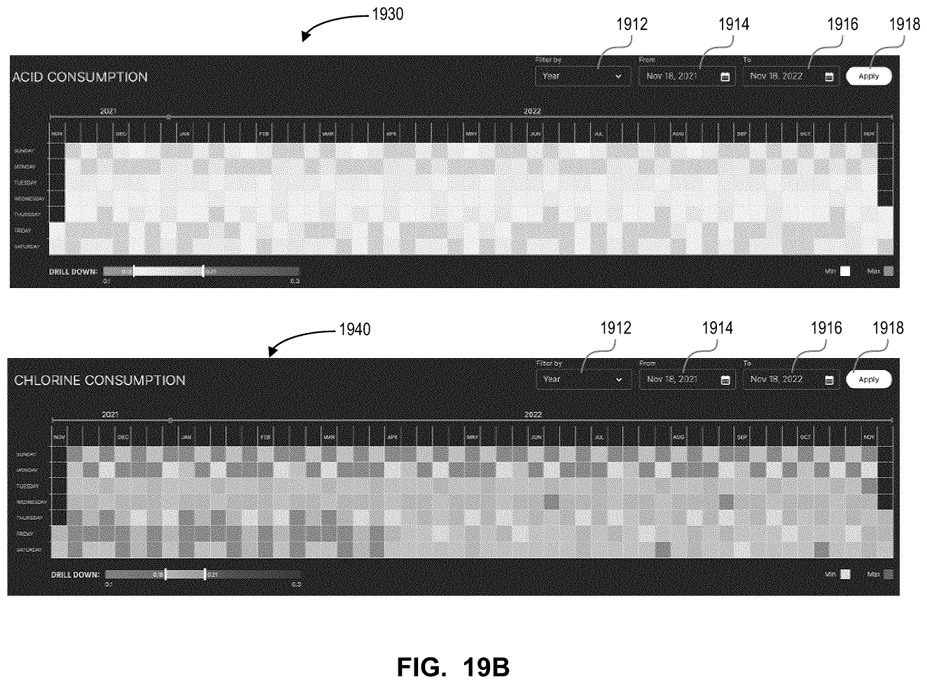

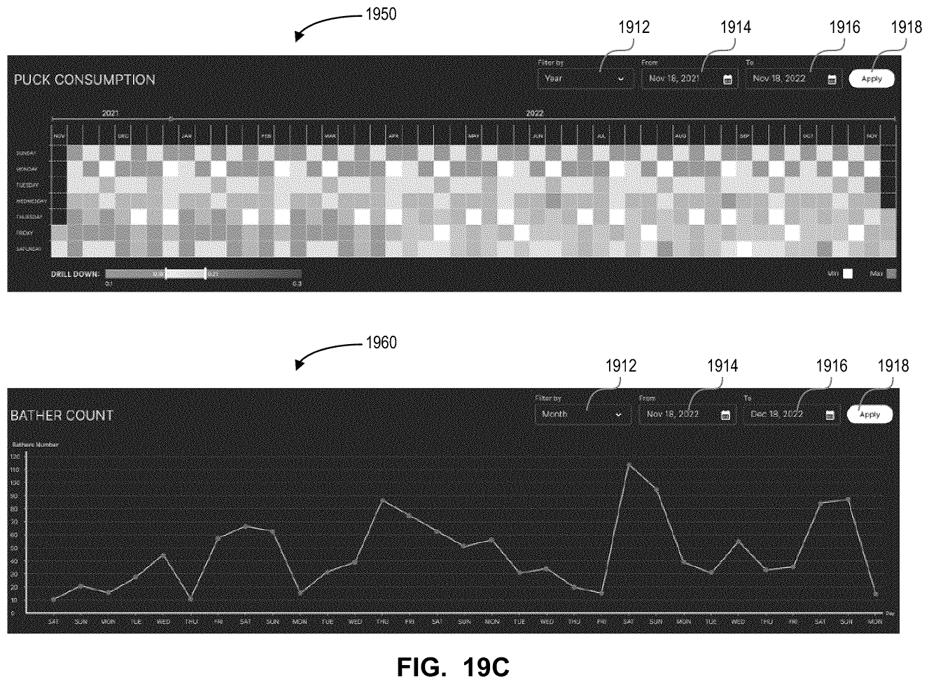

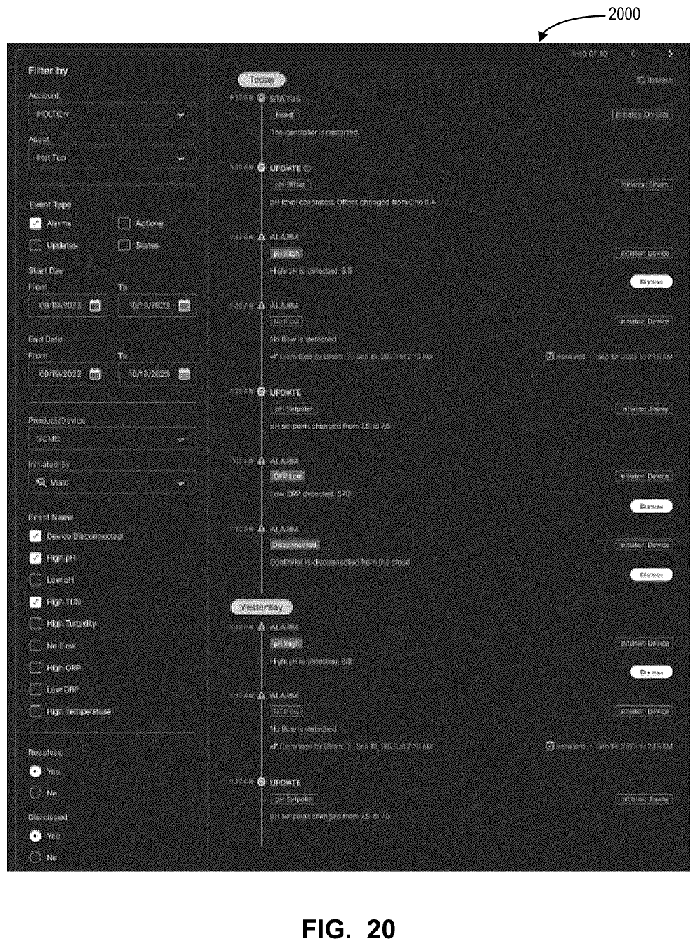

Turning now to FIGS. 19A, 19B, and 19C, examples of chart pages that are viewable through the interface 1500 are shown. The chart pages contain graphical plots or charts of various parameters including energy consumption 1910, water consumption 1920, acid consumption 1930, chlorine consumption 1940, puck consumption 1950, and bather count 1960. Each of the charts 1910-1960 includes a filter 1912, a start window 1914, an end window 1916, and an “apply” button 1918.

The energy consumption chart 1910 shows the energy consumption of the pool 102 for each day of the week for a span of time. The water consumption chart 1920 shows the water consumption of the pool 102 for each day of the week for a span of time. The acid consumption chart 1930 shows the acid consumption of the pool 102 for each day of the week for a span of time. The chlorine consumption chart 1940 shows the chlorine consumption of the pool 102 for each day of the week for a span of time. The puck consumption chart 1950 shows the puck consumption of the pool 102 for each day of the week for a span of time. The bather count chart 1950 shows the occupancy of the pool for each day over a span of time.

The span of time is user-adjustable, and is defined by user selection of an option from the filter 1912. For example, the time span may be in years, months, weeks, etc. Alternatively, the user may input a desired range, by entering a start date into the start window 1914 and an end date into the end window 1916. When the desired selection or time span is entered, pressing the “apply” button 1918 causes the corresponding chart to update with the energy consumption for the selected time span.

The energy consumption chart 1910, water consumption chart 1920, acid consumption chart 1930, chlorine consumption chart 1940, and puck consumption chart 1940 show the consumption for each day of the week in blocks that are allocated a colour. In the example shown, the darker the colour, the higher the consumption, and the lighter the colour, the lower the consumption. The bather count chart 1960 shows the occupancy for each day over time as a line graph.

Referring now to FIG. 20, an example notification log 2000 that is viewable through the interface 1500 is shown. The notification log 2000 is an automated list of all changes made to any of the general settings 1810, pH settings 1820, ORP settings 1830, temperature settings 1840, and alarm thresholds 1850, the status of components of any other systems described, or alarms triggered. The notification log 2000 can be filtered based on the type of notification. Notifications can be one of an alarm, an action, an update, or a state. The notification log 2000 can be filtered based on a time span. For example, a start date and an end date can be entered. The notification log 2000 can also be filtered based on entity, such as for example, the system 100, the chemical monitoring unit 106, the autonomous testing unit 150, or the system 1000.

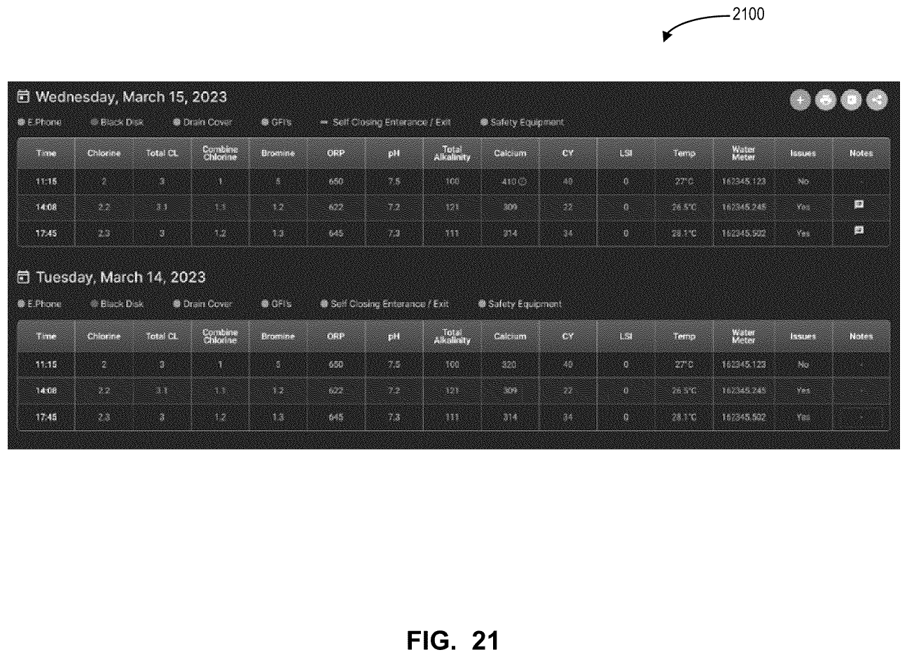

Referring now to FIG. 21, an example data log 2100 that is viewable through the interface 1500 is shown. The data log 2100 is an automated list of all values measured by any or all of the system 100, the chemical monitoring unit 106, the autonomous testing unit 150, or the system 1000. As new measurements are logged by each of the system 100, the chemical monitoring unit 106, the autonomous testing unit 150, or the system 1000, the values are added to the data log 2100. The user can view the most recent data alongside the data from previous measurements. The data log 2100 shows, for example, multiple measurements taken during a span of a day.

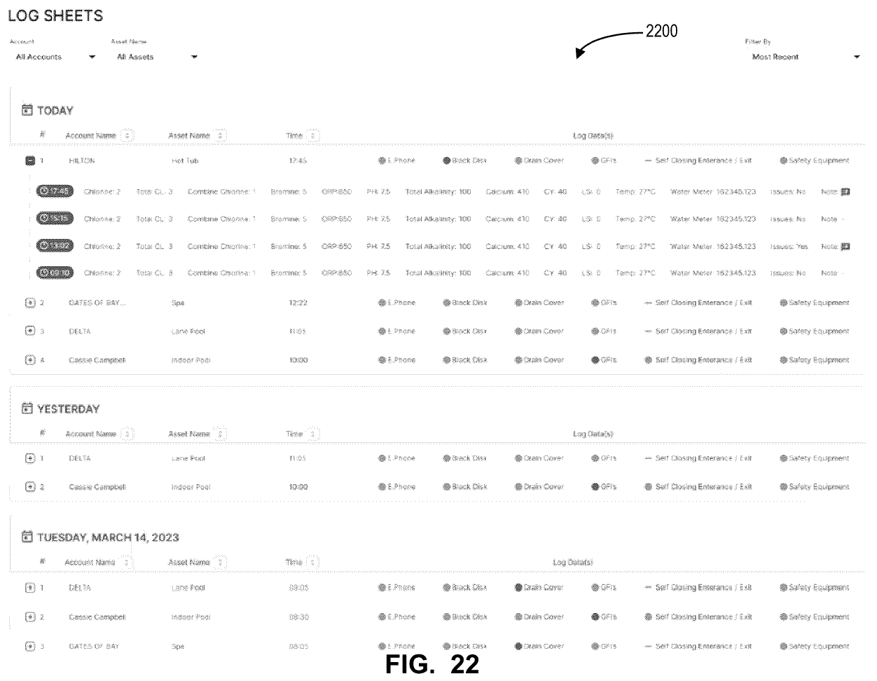

Referring now to FIG. 22, an example log sheet 2200 that is viewable through the interface 1500 is shown. Providing log sheets is a regulatory requirement in various states or provinces in the US and Canada. The log sheet 2200 provides a digital view of parameters in the pool 102, that may be gathered by each of the system 100, the chemical monitoring unit 106, the autonomous testing unit 150, or the system 1000. If at any time, any settings or recalibration of different components in any of the system 100, the chemical monitoring unit 106, the autonomous testing unit 150, or the system 1000 occur, they are reflected in the log sheet 2200. The log sheet 2200 may act as a feedback loop between each of the system 100, the chemical monitoring unit 106, the autonomous testing unit 150, or the system 1000 and any human/manual testing and checking of the pool 102 and its surrounding. Any discrepancies identified between the autonomous systems and manual checking reflected in the log sheet 2200 can be corrected and provide feedback on inconsistencies in manual observation.

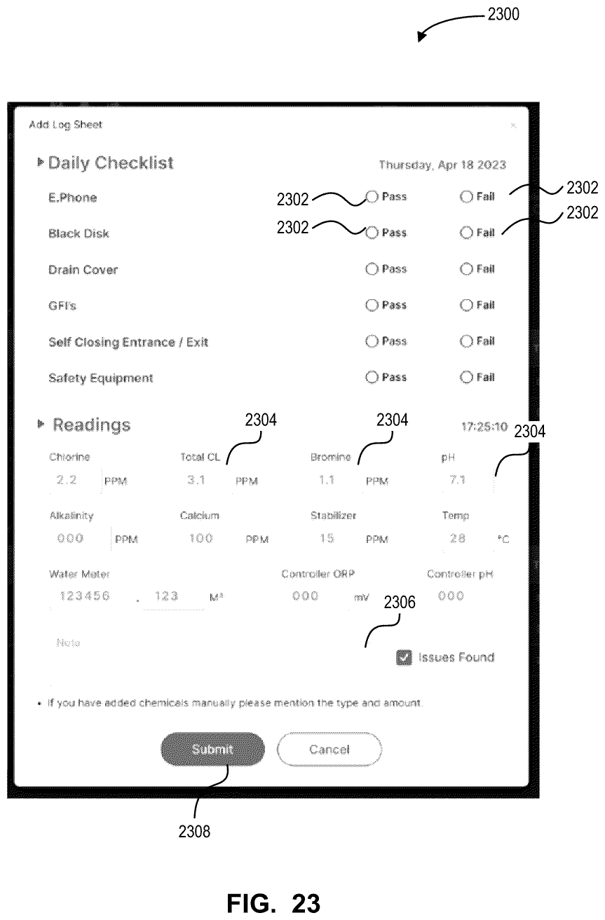

Referring now to FIG. 23, an example daily checklist 2300 that is viewable through the interface 1500 is shown. The daily checklist 2300 includes a number of selection boxes 2302, a number of entry windows 234, a note entry window 2306, and a “submit” button 2308. The daily checklist 2300 includes an automated list of parameters to be checked every day, that the user completes the daily checklist 2300 through the interface 1500. The completed daily checklist 2300 is stored.

The daily checklist 2300 includes, for example, pass or fail checks for several parameters such as the status of a drain cover in the pool, a status of the self closing entrance to the pool 102, the status of safety equipment in the pool 102 area. A user can select the selection box 2302 corresponding to “pass” or “fail” for each parameter. The parameters include, but are limited to, a status of an emergency phone, a status of the indicator 1020, a status of suction outlet drain covers, a status of safety equipment, and a status of GFI on the pool 102 deck. If the emergency phone does not work, has no dial tone, or does not connect to emergency services, the status is marked as a fail. If the indicator 1030 is not visible from the bottom of the pool 102, the status is marked as a fail. If the suction outlet drain covers that protect submerged suction ports in the pool are loose or missing, the status is marked as a fail. Due to strong suction levels, exposed ports pose high risks of entrapment, evisceration, and drowning. If safety equipment such as, buoys, spinal boards, Sheppard's hook, or other life saving devices are defective or not available, the status is marked as a fail. If the GFI is no ripping on the pool deck, the status is marked as a fail.

The daily checklist 2300 includes a number of readings that must be provided. These include values for, for example, chlorine, total chlorine, bromine, pH, alkalinity, calcium, stabilizer, temperature, water meter, ORP and pH controller. The values are entered by the user into a corresponding entry field 2304. If there are any other observations of note that the user deems appropriate for inclusion in the daily checklist 2300, these can be entered into a note entry window 2306. Once all selections and entries have been made, pressing a “submit” button 2308 submits the daily checklist 2300 for storage.

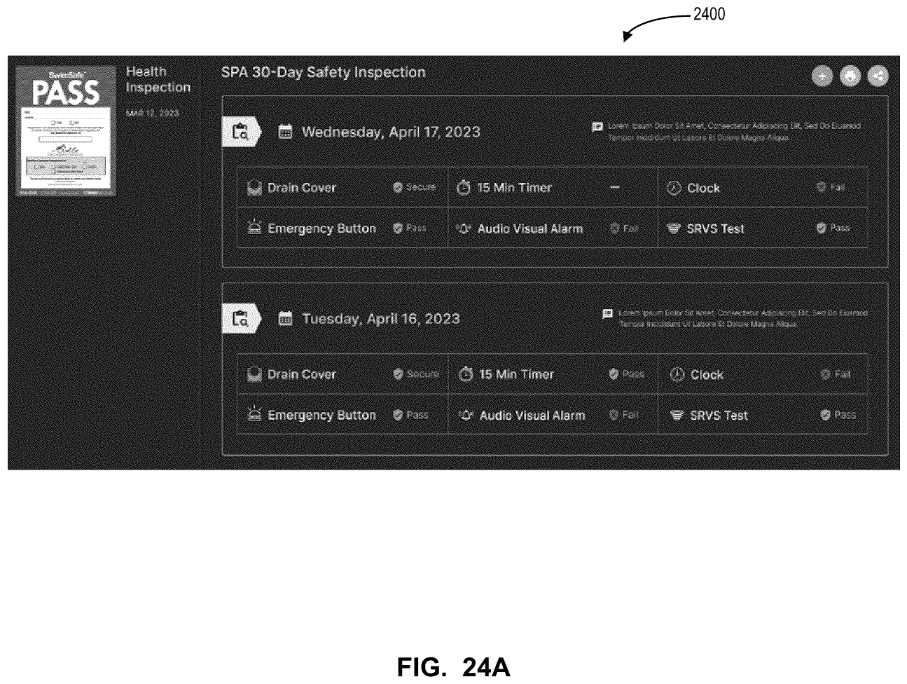

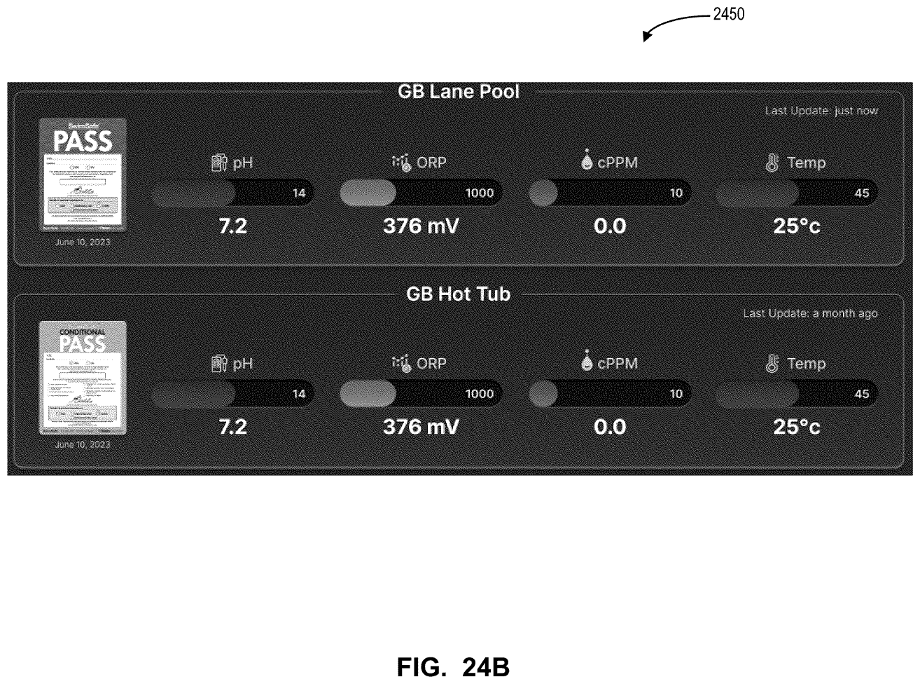

Referring now to FIGS. 24A and 24B, examples of safety inspection records that are viewable through the interface 1500 is shown. A first safety inspection 2400 shows the pass/fail status for the parameters in the daily checklist 2300 and the corresponding date. A second safety inspection record 2450 shows the values that were entered in daily checklist 2300 and when they were entered.

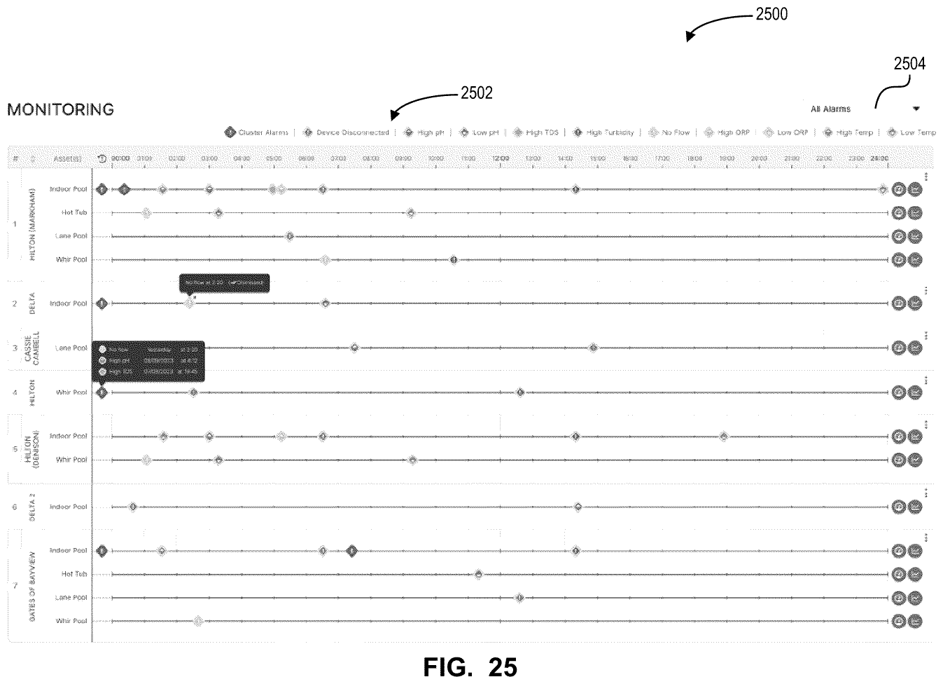

Referring now to FIG. 25, an example of a monitoring log 2500 that is viewable through the interface 1500 is shown. In some cases, the interface 1500 may provide data for more than one pool site, or there may be multiple pools in a location. The monitoring log 2500 provides a timeline illustration for each pool 102 or hot tub monitored, and when particular alarms are triggered. A legend 2502 is included for reference. The legend includes cluster alarms, a disconnected device alarm, high pH, low pH, high TDS, high turbidity, no flow, high ORP, low ORP, high temperature, and low temperature alarms. The monitoring log 2500 includes a filter 2504. The monitoring log 2500 can be filtered based on the type of alarm. When a particular type of alarm is selected in the filter 2504, the monitoring log 2500 is updated to show only events relating to that alarm.

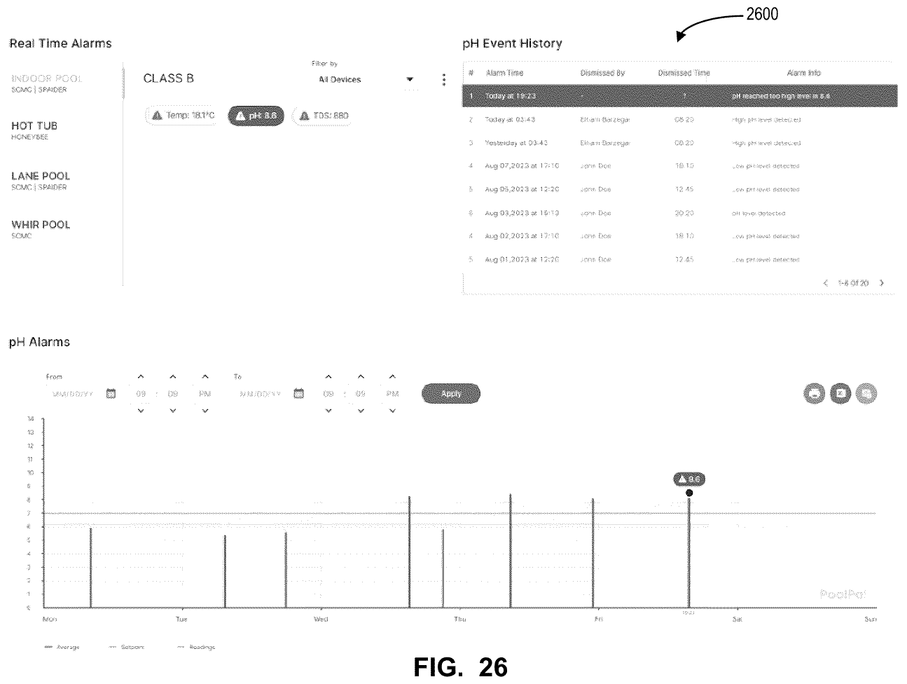

Referring now to FIG. 26, an example of an alarm log 2600 that is viewable through the interface 1500 is shown. Alarm log 2600 relates to overall monitoring of each of the system 100, the chemical monitoring unit 106, the autonomous testing unit 150, and the system 1000. The alarm log 2600 encapsulates and displays all components in each of the system 100, the chemical monitoring unit 106, the autonomous testing unit 150, and the system 1000 that are experiencing, or have unresolved, issues, errors, failures, out of bound readings, etc.

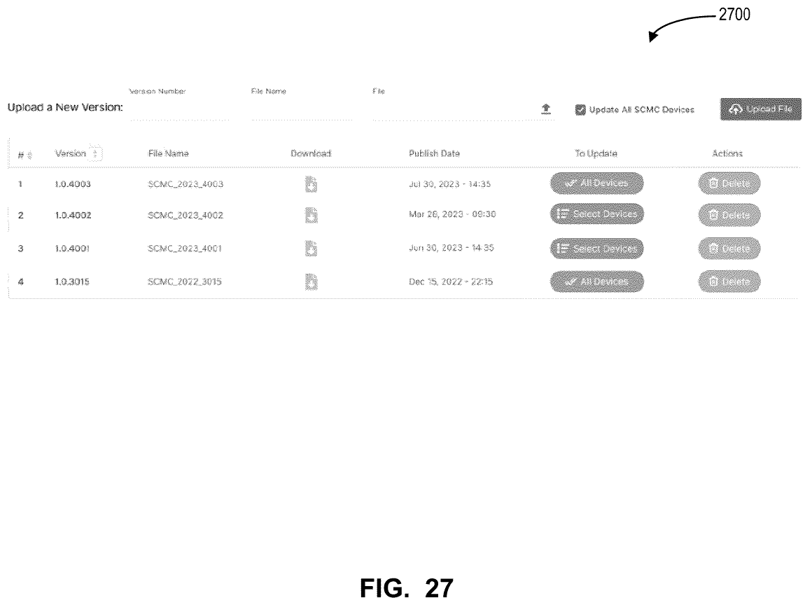

Referring now to FIG. 27, an example of a version history 2700 that is viewable through the interface 1500 is shown. The version history 2700 provides an update centre for over-the-air updating of devices individually, based on type, or as a group.

The system 100, the chemical monitoring unit 106, the autonomous testing unit 150, and the system 1000 are all either directly or indirectly connected to the cloud computer 130. The cloud computer 130 processes and stores all the data collected from the system 100, the chemical monitoring unit 106, the autonomous testing unit 150, and the system 1000 and makes it available to the user through the dashboard 1500. The data is autonomously collected and processed.

The described pool monitoring systems and methods enable those responsible for pool operations and maintenance to effectively and efficiently plan for pool maintenance. Pool operators and custodians are enabled to take proactive action with the provided estimations of expected equipment failure. The purchasing, application and storage of the chemical additives to the pool are optimized as the provided estimations and trends allow for adequate amounts of chemical additives to be purchased when required, rather than always maintaining a stockpile separate from the chemical and acid bins, that takes up space or stopping operations while procuring more chemical additives. Similarly, the maintenance of pool components can be optimized as replacement parts can be ordered in time rather than stopping operations while awaiting a new part.

Although the described pool monitoring systems and methods are defined in the context of commercial pools, the described systems and methods are applicable to other environments including spas, water treatment facilities, and industries where water may be stored and/or processed.

As used herein, an element or feature introduced in the singular and preceded by the word “a” or “an” should be understood as not necessarily excluding the plural of the elements or features. Further, references to “one example” or “one embodiment” are not intended to be interpreted as excluding the existence of additional examples or embodiments that also incorporate the described elements or features. Reference herein to “example” means that one or more feature, structure, element, component, characteristic and/or operational step described in connection with the example is included in at least one embodiment and/or implementation of the subject matter according to the subject disclosure. Thus, the phrases “an example,” “another example” and similar language throughout the subject disclosure may, but do not necessarily, refer to the same example. Further, the subject matter characterizing any one example may, but does not necessarily, include the subject matter characterizing any other example.

Unless explicitly stated to the contrary, examples or embodiments “comprising” or “having” or “including” an element or feature or a plurality of elements or features having a particular property may include additional elements or features not having that property. Also, it will be appreciated that the terms “comprises”, “has”, “includes” means “including but not limited to” and the terms “comprising”, “having” and “including” have equivalent meanings.

As used herein, the term “and/or” can include any and all combinations of one or more of the associated listed elements or features.

It will be understood that when an element or feature is referred to as being “on”, “attached” to, “affixed” to, “connected” to, “coupled” with, “contacting”, etc. another element or feature, that element or feature can be directly on, attached to, connected to, coupled with or contacting the other element or feature or intervening elements may also be present. In contrast, when an element or feature is referred to as being, for example, “directly on”, “directly attached” to, “directly affixed” to, “directly connected” to, “directly coupled” with or “directly contacting” another element of feature, there are no intervening elements or features present.

It will be understood that spatially relative terms, such as “under”, “below”, “lower”, “over”, “above”, “upper”, “front”, “back” and the like, may be used herein for ease of description to describe the relationship of an element or feature to another element or feature as illustrated in the figures. The spatially relative terms can however, encompass different orientations in use or operation in addition to the orientation depicted in the figures.

Reference herein to “configured” denotes an actual state of configuration that fundamentally ties the element or feature to the physical characteristics of the element or feature preceding the phrase “configured to.”

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of a lower-numbered item (e.g., a “first” item) and/or a higher-numbered item (e.g., a “third” item).

As used herein, the terms “approximately” and “about” represent an amount close to the stated amount that still performs the desired function or achieves the desired result. For example, the terms “approximately” and “about” may refer to an amount that is within engineering tolerances that would be readily appreciated by a person skilled in the art. Although embodiments have been described above with reference to the accompanying drawings, those of skill in the art will appreciate that variations and modifications may be made without departing from the scope thereof as defined by the appended claims.

What is claimed is:

1. A system for pool monitoring, the system comprising:
a plurality of sensors, each sensor being configured to monitor a parameter of a pool;
a control unit operatively connected to the plurality of sensors, the control unit being configured to determine a status of water in the pool based on a signal received one or more of the sensors, and
a cloud computer operatively connected to the control unit, the cloud computer configured to adjust a plurality of settings of the control unit; and
an end node computer operatively connected to the cloud computer,
the cloud computer being configured to transmit information received from each sensor and the status determined by the control unit to the end node computer for display.

2. The system of claim 1, wherein the plurality of sensors comprises at least two of: a water level sensor, a bin level sensor, a load cell sensor, a pump rpm sensor, a vibration sensor, a pressure sensor, a leak sensor, a water meter, a current meter, an ambient temperature sensor, and a relative humidity sensor.

3. The system of claim 1, further comprising a chemical monitoring unit, wherein the control unit is configured to determine the status of water in the pool based on information from the chemical monitoring unit, and wherein the cloud computer transmits information received from the chemical monitoring unit to the end node computer for display.

4. The system of claim 3, wherein the chemical monitoring unit comprises a pH sensor, an Oxidization Reduction Potential (ORP) sensor, a temperature sensor, a turbidity sensor, a total dissolved solids (TDS) sensor, and a water flow sensor.

5. The system of claim 2, wherein the control unit is operatively connected to a water pump, the pump rpm sensor and the water meter, the control unit being configured to control the water pump based on a signal received from the pump rpm sensor and the water meter.

6. The system of claim 2, wherein the control unit is operatively connected to the water level sensor and the leak sensor, the control unit being configured to determine a rate of water loss based on a signal from the water level sensor and the leak sensor.

7. The system of claim 2, further comprising an autonomous testing unit configured to test the water based on a predetermined schedule, and wherein the control unit is configured to determine the status of water in the pool based on information from the autonomous testing unit.

8. The system of claim 1, further comprising a machine learning component that, based on the transmitted information, is configured to generate a maintenance schedule.

9. A system for pool chemical monitoring, the system comprising:
a housing;
a plurality of sensors, each sensor of the plurality of sensors being configured to monitor a parameter of a pool;
a water flow monitor;
a processor, configured to receive a signal from each of the plurality of sensors and the water flow monitor to determine a status of water in the pool; and
a display, configured to receive the status from the processer and provide an indication of the status.

10. The system of claim 9, wherein the plurality of sensors comprises one or more of a pH sensor, an Oxidization Reduction Potential (ORP) sensor, a temperature sensor, a total dissolved solids (TDS) sensor, a turbidity sensor, and a water flow sensor.

11. The system of claim 9, wherein the housing is configured to receive a flow of water from the pool, the plurality of sensors being in fluid communication with the flow of water.

12. The system of claim 9, wherein the system further comprises a pH pump controller, operatively coupled to a pH pump.

13. An apparatus for autonomous water testing in a pool, the apparatus comprising:
a sample vessel situated in a moveable housing;
a plurality of valves, each valve coupled to a reagent supply or a water supply from the pool, and a flow meter; and
a microcontroller configured to control movement of the housing, a flow of reagent or water through the plurality of valves, and perform the water testing based on a predetermined schedule.

14. The apparatus of claim 13, wherein the plurality of valves comprises solenoid valves.

15. The apparatus of claim 13, wherein the microcontroller is configured to move the housing so that the sample vessel is in fluid communication with a source of tap water for washing the sample vessel.

16. A system for pool monitoring comprising:
at least one camera having a field of view of a pool and an area surrounding the pool;
an indicator disposed on a bottom surface of the pool;
a skimmer disposed on a wall of the pool; and
a processor operatively connected to the at least one camera and the alarm, the processor being configured to receive images of the pool captured by the at least one camera and determine one or more of:
at least one water parameter based on the received images with respect to at least one of the indicator and the skimmer;
an occupancy of the pool;
an occupancy of the area surrounding the pool; and
indications of pre-drowning of at least one occupant of the pool.

17. The system of claim 16, wherein the area surrounding the pool is divided into a plurality of deck occupancy zones.

18. The system of claim 16, wherein the pool is divided into one or more water occupancy zones.

19. The system of claim 16, wherein the at least one water parameter includes a water level and a water clarity.

20. The system of claim 16, wherein the processor determines the at least one water parameter based on a level of the water with respect to the water skimmer.
